SEQUENCE LISTING (10 sequences)

SEQ ID NO: 1 - RNA, 47 nt, Homo sapiens
gggaaauaagagagaaaagaagaguaagaagaaauauaagagccacc

SEQ ID NO: 2 - DNA, 1599 bp, Homo sapiens
atggctgtggagtcccagggcggacgcccacttgtcctgggcctgctgctgtgtgtgctgggcccagtggtgtcccatgctgggaagatactgttgatcccagtggatggcagccactggctgagcatgcttggggccatccagcagctgcagcagaggggacatgaaatagttgtcctagcacctgacgcctcgttgtacatcagagacggagcattttacaccttgaagacgtaccctgtgccattccaaagggaggatgtgaaagagtcttttgttagtctcgggcataatgtttttgagaatgattctttcctgcagcgtgtgatcaaaacatacaagaaaataaaaaaggactctgctatgcttttgtctggctgttcccacttactgcacaacaaggagctcatggcctccctggcagaaagcagctttgatgtcatgctgacggaccctttccttccttgcagccccatcgtggcccagtacctgtctctgcccactgtattcttcttgcatgcactgccatgcagcctggaatttgaggctacccagtgccccaacccattctcctacgtgcccaggcctctctcctctcattcagatcacatgaccttcctgcagcgggtgaagaacatgctcattgccttttcacagaactttctgtgcgacgtggtttattccccgtatgcaacccttgcctcagaattccttcagagagaggtgactgtccaggacctattgagctctgcatctgtctggctgtttagaagtgactttgtgaaggattaccctaggcccatcatgcccaatatggtttttgttggtggaatcaactgccttcaccaaaatccactatcccaggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccat

SEQ ID NO: 3 - RNA, 119 nt, Homo sapiens
ugauaauaggcuggagccucgguggccaugcuucuugccccuugggccuccccccagccccuccuccccuuccugcacccguacccccguggucuuugaauaaagucugagugggcggc

SEQ ID NO: 4 - protein, 533 aa, Homo sapiens
MAVESQGGRPLVLGLLLCVLGPVVSHAGKILLIPVDGSHWLSMLGAIQQLQQRGHEIVVLAPDASLYIRDGAFYTLKTYPVPFQREDVKESFVSLGHNVFENDSFLQRVIKTYKKIKKDSAMLLSGCSHLLHNKELMASLAESSFDVMLTDPFLPCSPIVAQYLSLPTVFFLHALPCSLEFEATQCPNPFSYVPRPLSSHSDHMTFLQRVKNMLIAFSQNFLCDVVYSPYATLASEFLQREVTVQDLLSSASVWLFRSDFVKDYPRPIMPNMVFVGGINCLHQNPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 5 - DNA, 1623 bp, artificial
  human UGT1A1 with a C-terminal FLAG tag
atggctgtggagtcccagggcggacgcccacttgtcctgggcctgctgctgtgtgtgctgggcccagtggtgtcccatgctgggaagatactgttgatcccagtggatggcagccactggctgagcatgcttggggccatccagcagctgcagcagaggggacatgaaatagttgtcctagcacctgacgcctcgttgtacatcagagacggagcattttacaccttgaagacgtaccctgtgccattccaaagggaggatgtgaaagagtcttttgttagtctcgggcataatgtttttgagaatgattctttcctgcagcgtgtgatcaaaacatacaagaaaataaaaaaggactctgctatgcttttgtctggctgttcccacttactgcacaacaaggagctcatggcctccctggcagaaagcagctttgatgtcatgctgacggaccctttccttccttgcagccccatcgtggcccagtacctgtctctgcccactgtattcttcttgcatgcactgccatgcagcctggaatttgaggctacccagtgccccaacccattctcctacgtgcccaggcctctctcctctcattcagatcacatgaccttcctgcagcgggtgaagaacatgctcattgccttttcacagaactttctgtgcgacgtggtttattccccgtatgcaacccttgcctcagaattccttcagagagaggtgactgtccaggacctattgagctctgcatctgtctggctgtttagaagtgactttgtgaaggattaccctaggcccatcatgcccaatatggtttttgttggtggaatcaactgccttcaccaaaatccactatcccaggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccatgactacaaagacgatgacgacaag

SEQ ID NO: 6 - protein, 541 aa, artificial
  human UGT1A1 with a C-terminal FLAG tag
MAVESQGGRPLVLGLLLCVLGPVVSHAGKILLIPVDGSHWLSMLGAIQQLQQRGHEIVVLAPDASLYIRDGAFYTLKTYPVPFQREDVKESFVSLGHNVFENDSFLQRVIKTYKKIKKDSAMLLSGCSHLLHNKELMASLAESSFDVMLTDPFLPCSPIVAQYLSLPTVFFLHALPCSLEFEATQCPNPFSYVPRPLSSHSDHMTFLQRVKNMLIAFSQNFLCDVVYSPYATLASEFLQREVTVQDLLSSASVWLFRSDFVKDYPRPIMPNMVFVGGINCLHQNPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTHDYKDDDDK

SEQ ID NO: 7 - DNA, 1605 bp, Rattus norvegicus
atgtccgtggtgtgccggagctcatgttcgcttctgcttcttccgtgccttctgctgtgtgtgttgggtccctctgcgtcccatgctgggaagctgttagtgatccccatagatggcagccactggctgagtatgctcggagttattcagcagctccagcaaaaggggcacgaagtggtggtcatagcacctgaagcttcgatacacataaaagaaggatcattttacactatgaggaagtaccctgtgccattccaaaatgaaaacgtgacagctgcttttgtggaacttgggcggagtgtctttgatcaagatccttttctgctgcgtgtggttaaaacatacaacaaagtcaaaagggactccagtatgctgctgtctggctgctcccaccttctgcacaatgccgagtttatggcctctctggaacaaagccactttgatgctctgctgacagaccctttccttccgtgtggctccattgtggcccagtacctgtctctgcctgctgtgtacttcttgaatgcattgccatgcagcctggatttggaagccacccaatgccctgctccgttgtcctacgtgcccaagagtttgtcctcgaacacagatcgcatgaacttcctgcagcgggtgaagaacatgattattgctttgacagagaactttctatgcagagtggtttactccccctatgggtcacttgccactgaaatcttacagaaagaggtgactgtcaaggaccttctgagtcctgcatctatctggctgatgagaaacgactttgtgaaagattaccccaggcccatcatgcccaacatggtttttattggtgggataaactgccttcagaaaaaagccctatcccaggaatttgaagcctatgtcaacgcctccggagaacatggcatcgtggttttctctttgggatccatggtctcagagattccagagaagaaagcgatggaaattgctgaggctttgggcagaattcctcagacggtcctgtggcgctacaccggaactagaccatcgaaccttgcaaagaacactattcttgtcaaatggctaccccaaaacgatctgcttggtcatccaaaggctcgggcgttcatcacacactccggttcccatggtatttatgaaggaatatgcaatggggttccaatggtgatgatgcccttgtttggtgatcagatggacaacgccaagcgcatggaaactcggggagctggggtgaccctgaatgtcctggaaatgactgccgatgatttggaaaacgcccttaaaactgtcatcaataacaagagttacaaggagaacatcatgcgcctctccagccttcacaaggaccgtcctatcgagcctctggacctggctgtgttctgggtggagtacgtgatgaggcacaagggggcgccacacctgcgccccgccgcccacgacctcacctggtaccagtaccactccttggacgtgattggctttctcctggccatcgtgttgacggtggtcttcattgtctataaaagttgtgcctatggctgccggaaatgctttgggggaaagggtcgagtgaagaaatcacacaaatccaagacccac

SEQ ID NO: 8 - protein, 535 aa, Rattus norvegicus
MSVVCRSSCSLLLLPCLLLCVLGPSASHAGKLLVIPIDGSHWLSMLGVIQQLQQKGHEVVVIAPEASIHIKEGSFYTMRKYPVPFQNENVTAAFVELGRSVFDQDPFLLRVVKTYNKVKRDSSMLLSGCSHLLHNAEFMASLEQSHFDALLTDPFLPCGSIVAQYLSLPAVYFLNALPCSLDLEATQCPAPLSYVPKSLSSNTDRMNFLQRVKNMIIALTENFLCRVVYSPYGSLATEILQKEVTVKDLLSPASIWLMRNDFVKDYPRPIMPNMVFIGGINCLQKKALSQEFEAYVNASGEHGIVVFSLGSMVSEIPEKKAMEIAEALGRIPQTVLWRYTGTRPSNLAKNTILVKWLPQNDLLGHPKARAFITHSGSHGIYEGICNGVPMVMMPLFGDQMDNAKRMETRGAGVTLNVLEMTADDLENALKTVINNKSYKENIMRLSSLHKDRPIEPLDLAVFWVEYVMRHKGAPHLRPAAHDLTWYQYHSLDVIGFLLAIVLTVVFIVYKSCAYGCRKCFGGKGRVKKSHKSKTH

SEQ ID NO: 9 - DNA, 1629 bp, artificial
  rat UGT1A1 with a C-terminal FLAG tag
atgtccgtggtgtgccggagctcatgttcgcttctgcttcttccgtgccttctgctgtgtgtgttgggtccctctgcgtcccatgctgggaagctgttagtgatccccatagatggcagccactggctgagtatgctcggagttattcagcagctccagcaaaaggggcacgaagtggtggtcatagcacctgaagcttcgatacacataaaagaaggatcattttacactatgaggaagtaccctgtgccattccaaaatgaaaacgtgacagctgcttttgtggaacttgggcggagtgtctttgatcaagatccttttctgctgcgtgtggttaaaacatacaacaaagtcaaaagggactccagtatgctgctgtctggctgctcccaccttctgcacaatgccgagtttatggcctctctggaacaaagccactttgatgctctgctgacagaccctttccttccgtgtggctccattgtggcccagtacctgtctctgcctgctgtgtacttcttgaatgcattgccatgcagcctggatttggaagccacccaatgccctgctccgttgtcctacgtgcccaagagtttgtcctcgaacacagatcgcatgaacttcctgcagcgggtgaagaacatgattattgctttgacagagaactttctatgcagagtggtttactccccctatgggtcacttgccactgaaatcttacagaaagaggtgactgtcaaggaccttctgagtcctgcatctatctggctgatgagaaacgactttgtgaaagattaccccaggcccatcatgcccaacatggtttttattggtgggataaactgccttcagaaaaaagccctatcccaggaatttgaagcctatgtcaacgcctccggagaacatggcatcgtggttttctctttgggatccatggtctcagagattccagagaagaaagcgatggaaattgctgaggctttgggcagaattcctcagacggtcctgtggcgctacaccggaactagaccatcgaaccttgcaaagaacactattcttgtcaaatggctaccccaaaacgatctgcttggtcatccaaaggctcgggcgttcatcacacactccggttcccatggtatttatgaaggaatatgcaatggggttccaatggtgatgatgcccttgtttggtgatcagatggacaacgccaagcgcatggaaactcggggagctggggtgaccctgaatgtcctggaaatgactgccgatgatttggaaaacgcccttaaaactgtcatcaataacaagagttacaaggagaacatcatgcgcctctccagccttcacaaggaccgtcctatcgagcctctggacctggctgtgttctgggtggagtacgtgatgaggcacaagggggcgccacacctgcgccccgccgcccacgacctcacctggtaccagtaccactccttggacgtgattggctttctcctggccatcgtgttgacggtggtcttcattgtctataaaagttgtgcctatggctgccggaaatgctttgggggaaagggtcgagtgaagaaatcacacaaatccaagacccacgactacaaagacgatgacgacaag

SEQ ID NO: 10 - protein, 543 aa, artificial
  rat UGT1A1 with a C-terminal FLAG tag
MSVVCRSSCSLLLLPCLLLCVLGPSASHAGKLLVIPIDGSHWLSMLGVIQQLQQKGHEVVVIAPEASIHIKEGSFYTMRKYPVPFQNENVTAAFVELGRSVFDQDPFLLRVVKTYNKVKRDSSMLLSGCSHLLHNAEFMASLEQSHFDALLTDPFLPCGSIVAQYLSLPAVYFLNALPCSLDLEATQCPAPLSYVPKSLSSNTDRMNFLQRVKNMIIALTENFLCRVVYSPYGSLATEILQKEVTVKDLLSPASIWLMRNDFVKDYPRPIMPNMVFIGGINCLQKKALSQEFEAYVNASGEHGIVVFSLGSMVSEIPEKKAMEIAEALGRIPQTVLWRYTGTRPSNLAKNTILVKWLPQNDLLGHPKARAFITHSGSHGIYEGICNGVPMVMMPLFGDQMDNAKRMETRGAGVTLNVLEMTADDLENALKTVINNKSYKENIMRLSSLHKDRPIEPLDLAVFWVEYVMRHKGAPHLRPAAHDLTWYQYHSLDVIGFLLAIVLTVVFIVYKSCAYGCRKCFGGKGRVKKSHKSKTHDYKDDDDK

(19) United States
(12) Patent Application Publication
(10) Pub. No.: US 2019/0307857 A1
(43) Pub. Date: Oct. 10, 2019

(54) MODIFIED mRNA ENCODING A URIDINE DIPHOPSPHATE GLUCURONOSYL TRANSFERASE AND USES THEREOF

(71) Applicant: ModernaTX, Inc., Cambridge, MA (US)

(72) Inventors: Melissa Lasaro, Sandy Hook, CT (US); David Osterman, Glastonbury, CT (US); Haren Vasavada, Hamden, CT (US); Christian Cobaugh, Newton Highlands, MA (US)

(73) Assignee: ModernaTX, Inc., Cambridge, MA (US)

(21) Appl. No.: 15/781,881

(22) PCT Filed: Dec. 9, 2016
(86) PCT No.: PCT/US2016/065814

§ 371 Date: Jun. 6, 2018

Related U.S. Application Data
(60) Provisional application No. 62/265,174, filed on Dec. 9, 2015.

(51) Int. Cl. A61K 38/45; A61K 31/7115; C12N 15/52; A61P 3/00; A61K 31/7105

(52) U.S. Cl. CPC A61K 38/45; A61K 31/7115; C12Y 204/01017; A61P 3/00; A61K 31/7105; C12N 15/52

(57) ABSTRACT

The invention relates to methods and compositions for treating a UDP glucuronosyltransferase family 1 deficiency based on mRNA therapy.

### BACKGROUND

Crigler-Najjar syndrome is a severe condition characterized by high levels of a toxic substance called bilirubin in the blood (hyperbilirubinemia). Bilirubin is produced when red blood cells are broken down. This substance is removed from the body only after it undergoes a chemical reaction in the liver, which converts the toxic form of bilirubin (unconjugated bilirubin) to a nontoxic form (conjugated bilirubin). Patients with Crigler-Najjar syndrome have a buildup of unconjugated bilirubin in their blood (unconjugated hyperbilirubinemia).

Bilirubin has an orange-yellow tint, and hyperbilirubinemia causes yellowing of the skin and whites of the eyes (jaundice). In Crigler-Najjar syndrome, jaundice is apparent at birth or in infancy. Severe unconjugated hyperbilirubinemia can lead to a condition called kernicterus, which is a form of brain damage caused by the accumulation of unconjugated bilirubin in the brain and nerve tissues. Babies with kernicterus are often extremely tired (lethargic) and may exhibit weak muscle tone (hypotonia). These babies may experience episodes of increased muscle tone (hypertonia) and arching of their backs. Kernicterus can lead to other neurological problems, including involuntary writhing movements of the body (choreoathetosis), hearing problems or intellectual disability.

As there is currently no effective treatment for the underlying genetic defect that leads to Crigler-Najjar and related diseases and disorders, development of a targeted therapeutic agent is needed.

### SUMMARY

Specific embodiments of the invention will become evident from the following more detailed description of certain embodiments and the claims.

In one embodiment, the disclosure is directed to a method of treating a disease or disorder associated with a uridine diphosphate glucuronosyltransferase family 1 deficiency in a subject comprising administering to the subject a therapeutically effective amount of a composition comprising a modified mRNA molecule encoding a uridine diphosphate glucuronosyltransferase 1 polypeptide or active fragment thereof. In a particular embodiment, the uridine diphosphate glucuronosyltransferase family 1 polypeptide is encoded by UGT1A1. In a particular embodiment, the uridine diphosphate glucuronosyltransferase family 1 polypeptide comprises an amino acid sequence that is at least about 80% identical to SEQ ID NO:4, at least 85% identical to SEQ ID NO:4, at least 90% identical to SEQ ID NO:4, at least 95% identical to SEQ ID NO:4, or an amino acid sequence identical to SEQ ID NO:4. In a particular embodiment, the modified mRNA molecule comprises a sequence complementary to a nucleotide sequence that is at least about 80% identical to SEQ ID NO:2, at least 85% identical to SEQ ID NO:2, at least 90% identical to SEQ ID NO:2, at least 95% identical to SEQ ID NO:2, or a sequence complementary to the nucleotide sequence of SEQ ID NO:2. In a particular embodiment, the uridine diphosphate glucuronosyltransferase family 1 deficiency is type 1 Crigler-Najjar syndrome, kernicterus or hyperbilirubinemia. In a particular embodiment, the modified mRNA molecule comprises at least one modified nucleoside selected from the group consisting of: pseudouridine, 1-methyl pseudouridine, N1-methyl pseudouridine, 5-methylcytidine, 5-methyluridine, 2′-O-methyluridine, 2-thiouridine and N⁶-methyladenosine. In a particular embodiment, the modified mRNA molecule comprises a poly(A) tail, a Kozak sequence, a 3′ untranslated region, a 5′ untranslated region or any combination thereof.

In one embodiment, the disclosure is directed to a pharmaceutical composition comprising a therapeutically effective amount of a modified mRNA molecule encoding a uridine diphosphate glucuronosyltransferase family 1 polypeptide or active fragment thereof, and a pharmaceutically acceptable carrier, diluent or excipient.

In one embodiment, the disclosure is directed to a pharmaceutical composition comprising a therapeutically effective amount of a modified mRNA molecule encoding a uridine diphosphate glucuronosyltransferase family 1 polypeptide or active fragment thereof formulated in a lipid nanoparticle carrier.

In one embodiment, the disclosure is directed to a method of reducing unconjugated bilirubin levels in a subject comprising administering a therapeutically effective amount of a modified mRNA capable of expressing a uridine diphosphate glucuronosyltransferase family 1 polypeptide or biologically active fragment thereof. In a particular embodiment, the uridine diphosphate glucuronosyltransferase family 1 polypeptide is encoded by UGT1A1. In a particular embodiment, the uridine diphosphate glucuronosyltransferase family 1 polypeptide comprises an amino acid sequence that is at least about 80% identical to SEQ ID NO:4, at least 85% identical to SEQ ID NO:4, at least 90% identical to SEQ ID NO:4, or an amino acid sequence that is at least 95% identical to SEQ ID NO:4.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows expression of human and rat UGT1A1 modRNAs in HeLa cells. The presence of human and rat UGT1A1 protein was detected with a cross-reactive anti-UGT1A1 antibody from cell lysates prepared 6, 24, 48 and 72 h after HeLa cells were transfected with hUGT1A1 modRNA or rUGT1A1 modRNA. As loading control, immunoblot analysis was performed using an anti-β-actin antibody. Recombinant human UGT1A1, human liver lysate and rat microsome preparation were used as positive control.

FIGS. 2A and 2B show expression and UGT1A1 enzyme activity of Gunn rat primary hepatocytes transfected with human and rat modRNA. FIG. 2A shows immunoblot analysis of cell lysates prepared 24 h after Gunn rat primary hepatocytes were transfected with hUGT1A1 modRNA (untagged and C-terminal FLAG) or rUGT1A1 modRNA(untagged and C-terminal FLAG). The presence of human and rat UGT1A1 protein was detected with human and rat selective anti-UGT1A1 antibodies. An anti-FLAG antibody was used to confirm the molecular weight and the presence of FLAG on the human and rat UGT1A1 constructs. Anti-β-actin antibody was used as loading control. Immunoblot results represent 1 out of 2 reproducible experiments. FIG. 2B shows enzyme activity-measured as the area under the peaks corresponding to mono- and di-glucuronides obtained from HPLC-UV chromatogram elution profiles after incubation of bilirubin with cell lysates after transfection. As mock control, cells were transfected with eGFP-modRNA.

FIGS. 3A and 3B show UGT1A1 expression and activity in CN1 patient-derived cells. FIG. 3A shows expression across three different lots of modRNA in fribroblasts derived from two different CN1 patients (UGT1A1 is present in both cells when transfected with any of the three different modRNA lots. No UGT1A1 expression was detected in the mock transfected cells. GAPDH was used as an expression control.

FIG. 3B is a plot showing UGT1A1 expression in CN1 patient-derived fibroblasts transfected buffer or three different lots of hUGT1A1 modRNA.

FIG. 4 shows hUGT1A1 protein expressed from modRNA targets the endoplasmic reticulum (ER). Immunocytochemistry against calnexin and human UGT1A1 proteins with Clone 9 cells transfected with hUGT1A1-modRNA. Clone 9 (K-9) is an epithelial cell line isolated from normal liver taken from a young male rat. Immunostainings were analyzed by fluorescent microscopy. hUGT1A1-modRNA transfected cells were fixed and incubated with corresponding primary antibodies. Immunoreactivity was visualized using Alexa Fluor® 488 anti-rabbit antibody solution (green) and Alexa Fluor® 594 anti-mouse antibody solution (red). The merge image with co-localization is showing in yellow. Cell nuclei are stained using DAPI (blue). Bar scale: 10 μm.

FIGS. 5A-D show expression, activity and localization of UGT1A1 in a Gunn rat model after administration of modRNA. FIG. 5A is a plot showing normalized levels of UGT1A1 after administration (0.2 mg/kg i.v.), which indicates a half-life of approximately 10 days post treatment. FIG. 5B shows UGT1A1 activity with regard to monoglucuronide levels (MGR) following administration of modRNA. FIG. 5C shows the activity of UGT1A1 in Gunn rats with respect to total plasma bilirubin levels following administration of modRNA. FIG. 5D shows localization of UGT1A1 in Gunn rats following administration of modRNA.

FIGS. 6A-C show hUGT1A1-modRNA chronic treatment results in sustained reduction of hyperbilirubinemia in Gunn rats. Three week old animals were dosed with modRNA at T₀, 14, 28, 42 and 58 days after the first dose. Arrows below graphs indicate time points of injections. Blood was collected once a week and one day before and one day after each subsequent dosing. FIG. 6A shows total plasma bilirubin levels in Gunn rats after Q2W injection (i.v.) of 0.1, 0.2 and 0.5 mg/kg hUGT1A1-modRNA. The range in wild-type rats treated with either PBS (n=6) or Luciferase-modRNA (n=6) is 0.129±0.313 mg/dL and 0.121±0.0095 mg/mL respectively. FIG. 6B is a comparison of the dose frequency effect on total plasma bilirubin levels in Gunn rats after Q2W and Q4W injection (i.v.) of 0.5 mg/kg hUGT1A1-modRNA. Red arrows below the graph indicate time points for modRNA treatment. FIG. 6C shows total bilirubin decay in naive animals and Luciferase-treated Gunn rats. For naive group, blood was collected once a week (n=14).

### DETAILED DESCRIPTION

Compositions and methods are described herein to treat or ameliorate a disease, disorder or condition associated with a uridine diphosphate glucuronosyltransferase family 1 (UGT1) deficiency, elevated unconjugated bilirubin, and elevated or deficient levels of molecular markers associated with a UGT1 deficiency, comprising administering to a subject a composition comprising a nucleic acid, e.g., a messenger RNA molecule, e.g., modified or unmodified, encoding a UGT1 polypeptide. As used herein, the term “messenger RNA” (mRNA) refers to a polynucleotide that encodes a polypeptide of interest and is capable of being translated to produce the encoded polypeptide of interest in vitro, in vivo, in situ or ex vivo. As used herein, “disease” refers to any deviation from the normal health of a subject and includes a state when disease symptoms are present, as well as conditions in which a deviation has occurred, but symptoms are not yet manifested (e.g., a predecease condition). As used herein, “treatment” or “treat” refer to both therapeutic treatment and prophylactic or preventative measures. Those in need of treatment include those having a disorder as well as those at risk for a disease or disorder, or those in whom the disorder is to be prevented.

Provided herein are nucleic acid molecules, including modified nucleic acid molecules, and methods of using the same. The nucleic acid molecules, including RNAs such as mRNAs, can comprise, for example, one or more modifications that improve properties of the molecule. Such improvements include, but are not limited to, increased stability and/or clearance in tissues, improved receptor uptake and/or kinetics, improved cellular access by the compositions, improved engagement with translational machinery, improved mRNA half-life, increased translation efficiency, improved immune evasion, improved protein production capacity, improved secretion efficiency, improved accessibility to circulation, improved protein half-life and/or modulation of a cell's status, improved function and/or improved activity.

The present disclosure provides compositions of nucleic acids capable of expressing or regulating protein expression of UGT1 or a biologically active fragment thereof in a target cell. Methods and processes of preparing and delivering such nucleic acid to a target cell are also provided. Kits and devices for the design, preparation, manufacture and formulation of such nucleic acids are also included in the instant disclosure.

The compositions provided herein are useful for treating a disease or disorder associated with a deficiency of UGT1 activity, such as, for example, Crigler-Najjar syndrome Type I (CN1). Crigler-Najjar syndrome is divided into two types. Type 1 (CN1) is very severe, and affected individuals can die in childhood due to kernicterus, although with proper treatment, they may survive longer. Type 2 (CN2) is less severe. People with CN2 are less likely to develop kernicterus, and most affected individuals survive into adulthood.

Preferred nucleic acids include, for example, polynucleotides, which further include, for example, ribonucleic acids (RNAs), deoxyribonucleic acids (DNAs), threose nucleic acids (TNAs; Yu, H. et al., Nat. Chem., 4:183-7, 2012), glycol nucleic acids (GNAs, for reviews see Ueda, N. et al., J. Heterocyclic Chem., 8:827-9, 1971; Zhang, L. et al., J. Am. Chem. Soc., 127:4174-5, 2005), peptide nucleic acids (PNAs, see Nielsen, P. et al., Science, 254:1497-500, 1991), locked nucleic acids (LNAs; Koshkin, A. et al., Tetrahedron, 54:3607-30, 1998), and other polynucleotides known in the art.

The nucleic acid molecule can be a messenger RNA (mRNA), e.g., a modified mRNA (“modRNA), which encodes, for example, a UGT1 (e.g., encoded by the UGT1A1 gene) or a biologically active fragment thereof. The mRNA can be delivered into a target cell, for example, to express a UGT1 or a biologically active fragment thereof. The mRNA can be translated in vivo, in situ or ex vivo.

The mRNA can be administered to an animal, e.g., a mammal (such as a human), to express a uridine diphosphate glucuronosyltransferase family 1 polypeptide or a biologically active fragment thereof. The mRNA provided is capable, for example, of treating or alleviating a symptom, a disease or a disorder associated with a deficiency of UGT1 activity, such as, for example, CN1.

### RNA Structure

Modified mRNA molecules are described herein that provide for a therapeutic tool for use in enzyme replacement therapy (ERT), e.g., for treating CN1 or a disease or condition associated with UGT1 deficiency. The terms “modified” or “modification” as used herein refer to an alteration of a nucleic acid residue that can be, for example, incorporated into a polynucleotide, e.g., an mRNA molecule, that can then be used for a therapeutic treatment. Modifications to an mRNA molecule can include, for example, physical or chemical modifications to a base, such as, for example, the depletion of a base or a chemical modification of a base, or sequence modifications to a nucleic acid sequence relative to a reference nucleic acid sequence.

Described herein are compositions for modulating the expression of a UGT1 or a biologically active fragment thereof in vitro or in vivo, e.g., in a target cell. The mRNA molecule can, for example, replace, increase or promote expression of such a UGT1 or biologically active fragment thereof. In some embodiments, the composition comprises an artificially synthesized or isolated nature RNA molecule with or without a transfer vehicle. An RNA molecule can comprise, for example, a sequential series of sequence elements, wherein, for example, sequence C comprises a nucleic acid sequence encoding a UGT1 or a biologically active fragment thereof. C may comprise, with or without a bridging linker (such as a peptide linker comprising at least one amino acid residue), one or more 5′ signal sequence(s). A sequence B, upstream of C, can comprise an optional flanking region comprising one or more complete or incomplete 5′ untranslated region (UTR) sequences. A sequence A, upstream of B, can comprise an optional 5′ terminal cap. A sequence D, downstream of C, can comprise an optional flanking region comprising one or more complete or incomplete 3′ UTR sequences. A sequence E, downstream of D, can comprise an optional flanking region comprising a 3′ tailing sequence. Bridging the 5′ terminus of C and the flanking sequence B is an optional first operational region. This first operational region traditionally comprises a start codon. The operational region can also comprise, for example, a translation initiation sequence or signal sequence. Bridging the 3′ end of C and the flanking region D is an optional second operational region. This second operational region can comprise, for example, a stop codon. The operational can also comprise a translation termination sequence or signal sequence. Multiple, serial stop codons can also be used. Sequence E can comprise a 3′ tail sequence, e.g., a poly-A tail.

UTRs are transcribed but not translated. The 5′ UTR starts at the transcription start site and continues to the start codon but does not include the start codon; whereas, the 3′ UTR starts immediately following the stop codon and continues until the transcriptional termination signal. Natural 5′ UTRs help translation initiation, and they comprise features such as, for example, Kozak sequences, which facilitate translation initiation by the ribosome for many genes. Kozak sequences have the consensus CCR(A/G)CCAUGG, where R is a purine (adenine or guanine) three bases upstream of the start codon (AUG), which is followed by another G.

3′ UTRs are rich in adenosines and uridines. These AU-rich signatures are particularly prevalent in genes with high rates of turnover. Based on their sequence features and functional properties, the AU-rich elements (AREs) can be separated into three classes-Class I AREs (such as those in c-Myc and MyoD) contain several dispersed copies of an AUUUA motif within U-rich regions; Class II AREs possess two or more overlapping UUAUUUA(U/A)(U/A) nonamers (molecules containing this type of ARE include GM-CSF and TNFα); Class III ARES are less well defined (these U-rich regions do not contain an AUUUA motif; c-Jun and myogenin are two examples of this class). Most proteins binding to the AREs destabilize the messenger, whereas members of the ELAV family, most notably HuR, increase the stability of mRNA. Engineering HuR specific binding site(s) into the 3′ UTR of the mRNA leads to HuR binding and thus, stabilization of the mRNA.

Introduction, removal or modification of 3′ UTR AREs can be used to modulate the stability of mRNA. When engineering specific mRNA, one or more copies of an ARE can be introduced to make such mRNA less stable and thereby curtail translation and decrease production of the resultant protein. Likewise, AREs can be identified and removed or mutated to increase the intracellular stability and thus increase translation and production of the resultant protein.

The 5′ cap structure of an mRNA is involved in nuclear export and mRNA stability in the cell. The cap binds to Cap Binding Protein (CBP), which is responsible for in vivo mRNA stability and translation competency through the interaction of CBP with poly-A binding protein to form the mature cyclic mRNA species. The cap further assists the removal of 5′ proximal introns during mRNA splicing. The mRNA molecules of the instant disclosure may be 5′ end capped to generate a 5′-ppp-5′-triphosphate linkage. The linkage site is between a terminal guanosine cap residue and the 5′-terminal transcribed sense nucleotide of the mRNA molecule. This 5′-guanylate cap may then be methylated to generate an N⁷-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

mRNA can be capped post-transcriptionally, for example, using enzymes to generate more authentic 5′ cap structures. As used herein, the phrase “more authentic” refers to a feature that closely mirrors or mimics, either structurally or functionally, a naturally occurring feature. That is, a “more authentic” feature is better representative of physiological cellular function and/or structure as compared to synthetic features or analogs. Non-limiting examples of more authentic 5′ cap structures are those that, among other things, have enhanced binding of CBPs, increased half-life, reduced susceptibility to 5′ endonucleases and/or reduced 5′ decapping, as compared to synthetic 5′ cap structures. Recombinant Vaccinia virus capping enzyme and recombinant 2′-O-methyltransferase, for example, can create a canonical 5′-5′-triphosphate linkage between the 5′ terminal nucleotide of an mRNA and a guanine cap nucleotide wherein the cap guanine contains an N7 methylation and the 5′ terminal nucleotide of the mRNA contains a 2′-O-methyl. Such a structure is termed the “Cap1” structure. This cap results in a higher translational competency and cellular stability and a reduced activation of cellular pro-inflammatory cytokines, as compared, for example, to other 5′-cap analog structures. Because the mRNA of the instant disclosure may be capped post-transcriptionally, and because this process is more efficient, nearly 100% of the mRNA may be capped. This is in contrast to the ˜80% capping rate when a cap analog is linked to an mRNA in the course of an in vitro transcription reaction.

Cap analogs can be used to modify the 5′ end of an mRNA molecule. Cap analogs, synthetic cap analogs, chemical caps, chemical cap analogs, or structural or functional cap analogs, differ from natural 5′ caps in their chemical structure, while still retaining cap function. Cap analogs can be chemically or enzymatically synthesized and/or linked to the mRNA, e.g., modRNA, described herein. The Anti-Reverse Cap Analog (ARCA), for example, contains two guanines linked by a 5′-5′-triphosphate group, wherein one guanine contains an N⁷ methyl group as well as a 3′-O-methyl group. Another exemplary cap is mCAP, which is similar to ARCA but has a 2′-O-methyl group on guanosine. Cap structures include, but are not limited to, 5′ triphosphate cap (5′-ppp), Guanosine-triphosphate Cap (5′-Gppp), 5′ N⁷-methylguanosine-triphosphate Cap (5′ N⁷-MeGppp, 7mGppp), 5′ Adenylated cap (rApp), 7mG(5′)ppp(5′)N, pN2p (cap 0), 7mG(5′)ppp(5′)N1mpNp (cap 1), and 7mG(5′)-ppp(5′)N1mpN2mp (cap 2) (Konarska, M. et al., Cell, 38:731-6, 1984; the entire contents of which are incorporated by reference). A 5′ terminal cap can further comprise a guanine analog. Useful guanine analogs include, but are not limited to, inosine, N¹-methyl-guanosine, 2′-fluoro-guanosine, 7-deaza-guanosine, 8-oxo-guanosine, 2-amino-guanosine, LNA-guanosine and 2-azido-guanosine.

### RNA Sequence

Described herein are modRNA sequences encoding a UGT1 or a biologically active fragment thereof, which is useful for, among other things, treating a disease or disorder associated with a deficiency of UGT1 activity, such as, for example, CN1. As used herein, a “biologically active fragment” refers to a portion of a molecule, e.g., a gene, coding sequence, mRNA, polypeptide or protein, which has a desired length or biological function. A biologically active fragment of a protein, for example, can be a fragment of the full-length protein that retains one or more biological activities of the protein. A biologically active fragment of an mRNA, for example, can be a fragment that, when translated, expresses a biologically active protein fragment. A biologically active mRNA fragment, furthermore, can comprise shortened versions of non-coding sequences, e.g., regulatory sequences, UTRs, etc. In general, a fragment of an enzyme or signaling molecule can be, for example, that portion(s) of the molecule that retains its signaling or enzymatic activity. A fragment of a gene or coding sequence, for example, can be that portion of the gene or coding sequence that produces an expression product fragment. As used herein, “gene” is a term used to describe a genetic element that gives rise to expression products (e.g., pre-mRNA, mRNA, polypeptides etc.). A fragment does not necessarily have to be defined functionally, as it can also refer to a portion of a molecule that is not the whole molecule, but has some desired characteristic or length (e.g., restriction fragments, amplification fragments, etc.).

Additional sequence modification, for example to the 3′ UTR, include the insertion of, for example, viral sequences such as the translation enhancer sequence of the barley yellow dwarf virus (BYDV-PAV), the Jaagsiekte sheep retrovirus (JSRV) and/or the Enzootic nasal tumor virus (PCT Pub. No. WO2012129648; herein incorporated by reference in its entirety).

modRNA described herein can comprise an internal ribosome entry site (IRES). IRESs play an important role in initiating protein synthesis in absence of the 5′ cap structure. An IRES can act as the sole ribosome binding site, or serve as one of multiple ribosome binding sites of an mRNA. An mRNA containing more than one functional ribosome binding site can encode several peptides or polypeptides that are translated independently by the ribosomes (“multicistronic nucleic acid molecules”). A modRNA can thus encode, for example, multiple portions or fragments of a UGT1 or a biologically active fragment thereof. Examples of IRES sequences that can be used include IRESs derived from, for example, picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) and cricket paralysis viruses (CrPV).

During RNA processing, a long chain of adenine nucleotides (poly-A tail) can be added to the mRNA molecule. The process, called polyadenylation, adds a poly-A tail that can be between, for example, about 100 and 250 residues long. In some embodiments, unique poly-A tail lengths provide certain advantages to the mRNA of the instant disclosure. Generally, the length of a poly-A tail is greater than 30 nucleotides in length (e.g., at least or greater than about 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the mRNA comprises a poly-A tail of a length from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000). In some embodiments, the poly-A tail is designed relative to the length of the overall mRNA. This design may be based on the length of the coding region, the length of a particular feature or region (such as the first or flanking regions), or based on the length of the ultimate product expressed from the mRNA. The poly-A tail can be, for example, 10, 20, 30, 40, 50, 60, 70, 80, 90 or 100% greater in length than the rest of the mRNA sequence. The poly-A tail can also be designed as a fraction of such mRNA.

mRNA can be linked together to the PABP (Poly-A binding protein) through the 3′ end using modified nucleotides at the 3′ terminus of the poly-A tail. In one embodiment, mRNA can include a poly-A tail-G-quartet. The G-quartet is a cyclic hydrogen bonded array of four guanine nucleotides that can be formed by G-rich sequences in both DNA and RNA. In this embodiment, the G-quartet is incorporated at the end of the poly-A tail.

Other RNA sequence modification elements and methods include a combination of nucleotide modifications abrogating mRNA interaction with Toll-like receptor 3 (TLR3), TLR7, TLR8 and retinoid-inducible gene 1 (RIG-1), resulting in low immunogenicity and higher stability in mice (Kormann, M. et al., Nat. Biotechnol., 29:154-7, 2011; the content of which is incorporated by reference herein in its entirety).

### UDP Glucuronosyltransferase Family 1

UGT1A1 is expressed from the UGT1A1 gene in humans. This gene encodes a UDP glucuronosyltransferase, an enzyme of the glucuronidation pathway that transforms small lipophilic molecules, such as steroids, bilirubin, hormones and drugs, into water-soluble, excretable metabolites. This gene is part of a complex locus that encodes several UDP-glucuronosyltransferases. The locus includes thirteen unique alternate first exons followed by four common exons. Four of the alternate first exons are considered pseudogenes. Each of the remaining nine 5′ exons may be spliced to the four common exons, resulting in nine proteins with different N-termini and identical C-termini. Each first exon encodes the substrate binding site, and is regulated by its own promoter. The preferred substrate of this enzyme is bilirubin, although it also has moderate activity with simple phenols, flavones, and C₁₈ steroids. Mutations in this gene result in Crigler-Najjar syndromes types I and II and in Gilbert syndrome.

Exemplary UGT1 sequences are shown below (including UTRs, cDNAs for ORFs and amino acid sequences from both human and rat). Modifications to the sequences can occur as described herein, for example, by using modified or non-naturally occurring uracil residues throughout the mRNA sequence.

hUGT1A1 modRNA

| mRNA Construct | |
|---|---|
| description | Human WT UGT1A1 with G5, C1 and T100 |
| 5′ UTR | GGGAAAUAAGAGAGAAAAGAAGAGUAAGAAGAAAUAUAAGAGCC |
| | ACC (SEQ ID NO: 1) |
| Corresponding | ATGGCTGTGGAGTCCCAGGGCGGACGCCCACTTGTCCTGGGCCT |
| nucleotide sequence | GCTGCTGTGTGTGCTGGGCCCAGTGGTGTCCCATGCTGGGAAGA |
| | TACTGTTGATCCCAGTGGATGGCAGCCACTGGCTGAGCATGCTT |
| | GGGGCCATCCAGCAGCTGCAGCAGAGGGGACATGAAATAGTTGT |
| | CCTAGCACCTGACGCCTCGTTGTACATCAGAGACGGAGCATTTT |
| | ACACCTTGAAGACGTACCCTGTGCCATTCCAAAGGGAGGATGTG |
| | AAAGAGTCTTTTGTTAGTCTCGGGCATAATGTTTTTGAGAATGA |
| | TTCTTTCCTGCAGCGTGTGATCAAAACATACAAGAAAATAAAAA |
| | AGGACTCTGCTATGCTTTTGTCTGGCTGTTCCCACTTACTGCAC |
| | AACAAGGAGCTCATGGCCTCCCTGGCAGAAAGCAGCTTTGATGT |
| | CATGCTGACGGACCCTTTCCTTCCTTGCAGCCCCATCGTGGCCC |
| | AGTACCTGTCTCTGCCCACTGTATTCTTCTTGCATGCACTGCCA |
| | TGCAGCCTGGAATTTGAGGCTACCCAGTGCCCCAACCCATTCTC |
| | CTACGTGCCCAGGCCTCTCTCCTCTCATTCAGATCACATGACCT |
| | TCCTGCAGCGGGTGAAGAACATGCTCATTGCCTTTTCACAGAAC |
| | TTTCTGTGCGACGTGGTTTATTCCCCGTATGCAACCCTTGCCTC |
| | AGAATTCCTTCAGAGAGAGGTGACTGTCCAGGACCTATTGAGCT |
| | CTGCATCTGTCTGGCTGTTTAGAAGTGACTTTGTGAAGGATTAC |
| | CCTAGGCCCATCATGCCCAATATGGTTTTTGTTGGTGGAATCAA |
| | CTGCCTTCACCAAAATCCACTATCCCAGGAATTTGAAGCCTACA |
| | TTAATGCTTCTGGAGAACATGGAATTGTGGTTTTCTCTTTGGGA |
| | TCAATGGTCTCAGAAATTCCAGAGAAGAAAGCTATGGCAATTGC |
| | TGATGCTTTGGGCAAAATCCCTCAGACAGTCCTGTGGCGGTACA |
| | CTGGAACCCGACCATCGAATCTTGCGAACAACACGATACTTGTT |
| | AAGTGGCTACCCCAAAACGATCTGCTTGGTCACCCGATGACCCG |
| | TGCCTTTATCACCCATGCTGGTTCCCATGGTGTTTATGAAAGCA |
| | TATGCAATGGCGTTCCCATGGTGATGATGCCCTTGTTTGGTGAT |
| | CAGATGGACAATGCAAAGCGCATGGAGACTAAGGGAGCTGGAGT |
| | GACCCTGAATGTTCTGGAAATGACTTCTGAAGATTTAGAAAATG |
| | CTCTAAAAGCAGTCATCAATGACAAAAGTTACAAGGAGAACATC |
| | ATGCGCCTCTCCAGCCTTCACAAGGACCGCCCGGTGGAGCCGCT |
| | GGACCTGGCCGTGTTCTGGGTGGAGTTTGTGATGAGGCACAAGG |
| | GCGCGCCACACCTGCGCCCCGCAGCCCACGACCTCACCTGGTAC |
| | CAGTACCATTCCTTGGACGTGATTGGTTTCCTCTTGGCCGTCGT |
| | GCTGACAGTGGCCTTCATCACCTTTAAATGTTGTGCTTATGGCT |
| | ACCGGAAATGCTTGGGGAAAAAAGGGCGAGTTAAGAAAGCCCAC |
| | AAATCCAAGACCCAT (SEQ ID NO: 2) |
| 3′ UTR | UGAUAAUAGGCUGGAGCCUCGGUGGCCAUGCUUCUUGCCCCUUG |
| | GGCCUCCCCCCAGCCCCUCCUCCCCUUCCUGCACCCGUACCCCC |
| | GUGGUCUUUGAAUAAAGUCUGAGUGGGCGGC (SEQ ID |
| | NO: 3) |
| Corresponding amino | MAVESQGGRPLVLGLLLCVLGPVVSHAGKILLIPVDGSHWLSML |
| acid sequence | GAIQQLQQRGHEIVVLAPDASLYIRDGAFYTLKTYPVPFQREDV |
| | KESFVSLGHNVFENDSFLQRVIKTYKKIKKDSAMLLSGCSHLLH |
| | NKELMASLAESSFDVMLTDPFLPCSPIVAQYLSLPTVFFLHALP |
| | CSLEFEATQCPNPFSYVPRPLSSHSDHMTFLQRVKNMLIAFSQN |
| | FLCDVVYSPYATLASEFLQREVTVQDLLSSASVWLFRSDFVKDY |
| | PRPIMPNMVFVGGINCLHQNPLSQEFEAYINASGEHGIVVFSLG |
| | SMVSEIPEKKAMAIADALGKIPQTVLWRYTGTRPSNLANNTILV |
| | KWLPQNDLLGHPMTRAFITHAGSHGVYESICNGVPMVMMPLFGD |
| | QMDNAKRMETKGAGVTLNVLEMTSEDLENALKAVINDKSYKENI |
| | MRLSSLHKDRPVEPLDLAVFWVEFVMRHKGAPHLRPAAHDLTWY |
| | QYHSLDVIGFLLAVVLTVAFITFKCCAYGYRKCLGKKGRVKKAH |
| | KSKTH (SEQ ID NO: 4) |
| Play tail | 100 nt |

hUGT1A1-FLAG (C-terminal) modRNA

| mRNA Construct | Human WT UGT1A1 + FLAG tag at the C-terminal |
|---|---|
| description | with G5, C1 and T100 |
| Corresponding | ATGGCTGTGGAGTCCCAGGGCGGACGCCCACTTGTCCTGGGCCT |
| nucleotide sequence | GCTGCTGTGTGTGCTGGGCCCAGTGGTGTCCCATGCTGGGAAGA |
| | TACTGTTGATCCCAGTGGATGGCAGCCACTGGCTGAGCATGCTT |
| | GGGGCCATCCAGCAGCTGCAGCAGAGGGGACATGAAATAGTTGT |
| | CCTAGCACCTGACGCCTCGTTGTACATCAGAGACGGAGCATTTT |
| | ACACCTTGAAGACGTACCCTGTGCCATTCCAAAGGGAGGATGTG |
| | AAAGAGTCTTTTGTTAGTCTCGGGCATAATGTTTTTGAGAATGA |
| | TTCTTTCCTGCAGCGTGTGATCAAAACATACAAGAAAATAAAAA |
| | AGGACTCTGCTATGCTTTTGTCTGGCTGTTCCCACTTACTGCAC |
| | AACAAGGAGCTCATGGCCTCCCTGGCAGAAAGCAGCTTTGATGT |
| | CATGCTGACGGACCCTTTCCTTCCTTGCAGCCCCATCGTGGCCC |
| | AGTACCTGTCTCTGCCCACTGTATTCTTCTTGCATGCACTGCCA |
| | TGCAGCCTGGAATTTGAGGCTACCCAGTGCCCCAACCCATTCTC |
| | CTACGTGCCCAGGCCTCTCTCCTCTCATTCAGATCACATGACCT |
| | TCCTGCAGCGGGTGAAGAACATGCTCATTGCCTTTTCACAGAAC |
| | TTTCTGTGCGACGTGGTTTATTCCCCGTATGCAACCCTTGCCTC |
| | AGAATTCCTTCAGAGAGAGGTGACTGTCCAGGACCTATTGAGCT |
| | CTGCATCTGTCTGGCTGTTTAGAAGTGACTTTGTGAAGGATTAC |
| | CCTAGGCCCATCATGCCCAATATGGTTTTTGTTGGTGGAATCAA |
| | CTGCCTTCACCAAAATCCACTATCCCAGGAATTTGAAGCCTACA |
| | TTAATGCTTCTGGAGAACATGGAATTGTGGTTTTCTCTTTGGGA |
| | TCAATGGTCTCAGAAATTCCAGAGAAGAAAGCTATGGCAATTGC |
| | TGATGCTTTGGGCAAAATCCCTCAGACAGTCCTGTGGCGGTACA |
| | CTGGAACCCGACCATCGAATCTTGCGAACAACACGATACTTGTT |
| | AAGTGGCTACCCCAAAACGATCTGCTTGGTCACCCGATGACCCG |
| | TGCCTTTATCACCCATGCTGGTTCCCATGGTGTTTATGAAAGCA |
| | TATGCAATGGCGTTCCCATGGTGATGATGCCCTTGTTTGGTGAT |
| | CAGATGGACAATGCAAAGCGCATGGAGACTAAGGGAGCTGGAGT |
| | GACCCTGAATGTTCTGGAAATGACTTCTGAAGATTTAGAAAATG |
| | CTCTAAAAGCAGTCATCAATGACAAAAGTTACAAGGAGAACATC |
| | ATGCGCCTCTCCAGCCTTCACAAGGACCGCCCGGTGGAGCCGCT |
| | GGACCTGGCCGTGTTCTGGGTGGAGTTTGTGATGAGGCACAAGG |
| | GCGCGCCACACCTGCGCCCCGCAGCCCACGACCTCACCTGGTAC |
| | CAGTACCATTCCTTGGACGTGATTGGTTTCCTCTTGGCCGTCGT |
| | GCTGACAGTGGCCTTCATCACCTTTAAATGTTGTGCTTATGGCT |
| | ACCGGAAATGCTTGGGGAAAAAAGGGCGAGTTAAGAAAGCCCAC |
| | AAATCCAAGACCCATGACTACAAAGACGATGACGACAAG (SEQ |
| | ID NO: 5) |
| Corresponding amino | MAVESQGGRPLVLGLLLCVLGPVVSHAGKILLIPVDGSHWLSML |
| acid sequence | GAIQQLQQRGHEIVVLAPDASLYIRDGAFYTLKTYPVPFQREDV |
| | KESFVSLGHNVFENDSFLQRVIKTYKKIKKDSAMLLSGCSHLLH |
| | NKELMASLAESSFDVMLTDPFLPCSPIVAQYLSLPTVFFLHALP |
| | CSLEFEATQCPNPFSYVPRPLSSHSDHMTFLQRVKNMLIAFSQN |
| | FLCDVVYSPYATLASEFLQREVTVQDLLSSASVWLFRSDFVKDY |
| | PRPIMPNMVFVGGINCLHQNPLSQEFEAYINASGEHGIVVFSLG |
| | SMVSEIPEKKAMAIADALGKIPQTVLWRYTGTRPSNLANNTILV |
| | KWLPQNDLLGHPMTRAFITHAGSHGVYESICNGVPMVMMPLFGD |
| | QMDNAKRMETKGAGVTLNVLEMTSEDLENALKAVINDKSYKENI |
| | MRLSSLHKDRPVEPLDLAVFWVEFVMRHKGAPHLRPAAHDLTWY |
| | QYHSLDVIGFLLAVVLTVAFITFKCCAYGYRKCLGKKGRVKKAH |
| | KSKTHDYKDDDDK (SEQ ID NO: 6) |

rUGT1A1 modRNA

| mRNA Construct | |
|---|---|
| description | rat WT UGT1A1 with G5, C1 and T100 |
| Corresponding | ATGTCCGTGGTGTGCCGGAGCTCATGTTCGCTTCTGCTTCTTCC |
| nucleotide sequence | GTGCCTTCTGCTGTGTGTGTTGGGTCCCTCTGCGTCCCATGCTG |
| | GGAAGCTGTTAGTGATCCCCATAGATGGCAGCCACTGGCTGAGT |
| | ATGCTCGGAGTTATTCAGCAGCTCCAGCAAAAGGGGCACGAAGT |
| | GGTGGTCATAGCACCTGAAGCTTCGATACACATAAAAGAAGGAT |
| | CATTTTACACTATGAGGAAGTACCCTGTGCCATTCCAAAATGAA |
| | AACGTGACAGCTGCTTTTGTGGAACTTGGGCGGAGTGTCTTTGA |
| | TCAAGATCCTTTTCTGCTGCGTGTGGTTAAAACATACAACAAAG |
| | TCAAAAGGGACTCCAGTATGCTGCTGTCTGGCTGCTCCCACCTT |
| | CTGCACAATGCCGAGTTTATGGCCTCTCTGGAACAAAGCCACTT |
| | TGATGCTCTGCTGACAGACCCTTTCCTTCCGTGTGGCTCCATTG |
| | TGGCCCAGTACCTGTCTCTGCCTGCTGTGTACTTCTTGAATGCA |
| | TTGCCATGCAGCCTGGATTTGGAAGCCACCCAATGCCCTGCTCC |
| | GTTGTCCTACGTGCCCAAGAGTTTGTCCTCGAACACAGATCGCA |
| | TGAACTTCCTGCAGCGGGTGAAGAACATGATTATTGCTTTGACA |
| | GAGAACTTTCTATGCAGAGTGGTTTACTCCCCCTATGGGTCACT |
| | TGCCACTGAAATCTTACAGAAAGAGGTGACTGTCAAGGACCTTC |
| | TGAGTCCTGCATCTATCTGGCTGATGAGAAACGACTTTGTGAAA |
| | GATTACCCCAGGCCCATCATGCCCAACATGGTTTTTATTGGTGG |
| | GATAAACTGCCTTCAGAAAAAAGCCCTATCCCAGGAATTTGAAG |
| | CCTATGTCAACGCCTCCGGAGAACATGGCATCGTGGTTTTCTCT |
| | TTGGGATCCATGGTCTCAGAGATTCCAGAGAAGAAAGCGATGGA |
| | AATTGCTGAGGCTTTGGGCAGAATTCCTCAGACGGTCCTGTGGC |
| | GCTACACCGGAACTAGACCATCGAACCTTGCAAAGAACACTATT |
| | CTTGTCAAATGGCTACCCCAAAACGATCTGCTTGGTCATCCAAA |
| | GGCTCGGGCGTTCATCACACACTCCGGTTCCCATGGTATTTATG |
| | AAGGAATATGCAATGGGGTTCCAATGGTGATGATGCCCTTGTTT |
| | GGTGATCAGATGGACAACGCCAAGCGCATGGAAACTCGGGGAGC |
| | TGGGGTGACCCTGAATGTCCTGGAAATGACTGCCGATGATTTGG |
| | AAAACGCCCTTAAAACTGTCATCAATAACAAGAGTTACAAGGAG |
| | AACATCATGCGCCTCTCCAGCCTTCACAAGGACCGTCCTATCGA |
| | GCCTCTGGACCTGGCTGTGTTCTGGGTGGAGTACGTGATGAGGC |
| | ACAAGGGGGCGCCACACCTGCGCCCCGCCGCCCACGACCTCACC |
| | TGGTACCAGTACCACTCCTTGGACGTGATTGGCTTTCTCCTGGC |
| | CATCGTGTTGACGGTGGTCTTCATTGTCTATAAAAGTTGTGCCT |
| | ATGGCTGCCGGAAATGCTTTGGGGGAAAGGGTCGAGTGAAGAAA |
| | TCACACAAATCCAAGACCCAC (SEQ ID NO: 7) |
| Corresponding amino | MSVVCRSSCSLLLLPCLLLCVLGPSASHAGKLLVIPIDGSHWLS |
| acid sequence | MLGVIQQLQQKGHEVVVIAPEASIHIKEGSFYTMRKYPVPFQNE |
| | NVTAAFVELGRSVFDQDPFLLRVVKTYNKVKRDSSMLLSGCSHL |
| | LHNAEFMASLEQSHFDALLTDPFLPCGSIVAQYLSLPAVYFLNA |
| | LPCSLDLEATQCPAPLSYVPKSLSSNTDRMNFLQRVKNMIIALT |
| | ENFLCRVVYSPYGSLATEILQKEVTVKDLLSPASIWLMRNDFVK |
| | DYPRPIMPNMVFIGGINCLQKKALSQEFEAYVNASGEHGIVVFS |
| | LGSMVSEIPEKKAMEIAEALGRIPQTVLWRYTGTRPSNLAKNTI |
| | LVKWLPQNDLLGHPKARAFITHSGSHGIYEGICNGVPMVMMPLF |
| | GDQMDNAKRMETRGAGVTLNVLEMTADDLENALKTVINNKSYKE |
| | NIMRLSSLHKDRPIEPLDLAVFWVEYVMRHKGAPHLRPAAHDLT |
| | WYQYHSLDVIGFLLAIVLTVVFIVYKSCAYGCRKCFGGKGRVKK |
| | SHKSKTH (SEQ ID NO: 8) |

rUGT1A1-FLAG (C-terminal) modRNA

| mRNA Construct | rat WT UGT1A1 + FLAG tag at the C-terminal |
|---|---|
| description | with G5, C1 and T100 |
| Corresponding | ATGTCCGTGGTGTGCCGGAGCTCATGTTCGCTTCTGCTTCTTCC |
| nucleotide sequence | GTGCCTTCTGCTGTGTGTGTTGGGTCCCTCTGCGTCCCATGCTG |
| | GGAAGCTGTTAGTGATCCCCATAGATGGCAGCCACTGGCTGAGT |
| | ATGCTCGGAGTTATTCAGCAGCTCCAGCAAAAGGGGCACGAAGT |
| | GGTGGTCATAGCACCTGAAGCTTCGATACACATAAAAGAAGGAT |
| | CATTTTACACTATGAGGAAGTACCCTGTGCCATTCCAAAATGAA |
| | AACGTGACAGCTGCTTTTGTGGAACTTGGGCGGAGTGTCTTTGA |
| | TCAAGATCCTTTTCTGCTGCGTGTGGTTAAAACATACAACAAAG |
| | TCAAAAGGGACTCCAGTATGCTGCTGTCTGGCTGCTCCCACCTT |
| | CTGCACAATGCCGAGTTTATGGCCTCTCTGGAACAAAGCCACTT |
| | TGATGCTCTGCTGACAGACCCTTTCCTTCCGTGTGGCTCCATTG |
| | TGGCCCAGTACCTGTCTCTGCCTGCTGTGTACTTCTTGAATGCA |
| | TTGCCATGCAGCCTGGATTTGGAAGCCACCCAATGCCCTGCTCC |
| | GTTGTCCTACGTGCCCAAGAGTTTGTCCTCGAACACAGATCGCA |
| | TGAACTTCCTGCAGCGGGTGAAGAACATGATTATTGCTTTGACA |
| | GAGAACTTTCTATGCAGAGTGGTTTACTCCCCCTATGGGTCACT |
| | TGCCACTGAAATCTTACAGAAAGAGGTGACTGTCAAGGACCTTC |
| | TGAGTCCTGCATCTATCTGGCTGATGAGAAACGACTTTGTGAAA |
| | GATTACCCCAGGCCCATCATGCCCAACATGGTTTTTATTGGTGG |
| | GATAAACTGCCTTCAGAAAAAAGCCCTATCCCAGGAATTTGAAG |
| | CCTATGTCAACGCCTCCGGAGAACATGGCATCGTGGTTTTCTCT |
| | TTGGGATCCATGGTCTCAGAGATTCCAGAGAAGAAAGCGATGGA |
| | AATTGCTGAGGCTTTGGGCAGAATTCCTCAGACGGTCCTGTGGC |
| | GCTACACCGGAACTAGACCATCGAACCTTGCAAAGAACACTATT |
| | CTTGTCAAATGGCTACCCCAAAACGATCTGCTTGGTCATCCAAA |
| | GGCTCGGGCGTTCATCACACACTCCGGTTCCCATGGTATTTATG |
| | AAGGAATATGCAATGGGGTTCCAATGGTGATGATGCCCTTGTTT |
| | GGTGATCAGATGGACAACGCCAAGCGCATGGAAACTCGGGGAGC |
| | TGGGGTGACCCTGAATGTCCTGGAAATGACTGCCGATGATTTGG |
| | AAAACGCCCTTAAAACTGTCATCAATAACAAGAGTTACAAGGAG |
| | AACATCATGCGCCTCTCCAGCCTTCACAAGGACCGTCCTATCGA |
| | GCCTCTGGACCTGGCTGTGTTCTGGGTGGAGTACGTGATGAGGC |
| | ACAAGGGGGCGCCACACCTGCGCCCCGCCGCCCACGACCTCACC |
| | TGGTACCAGTACCACTCCTTGGACGTGATTGGCTTTCTCCTGGC |
| | CATCGTGTTGACGGTGGTCTTCATTGTCTATAAAAGTTGTGCCT |
| | ATGGCTGCCGGAAATGCTTTGGGGGAAAGGGTCGAGTGAAGAAA |
| | TCACACAAATCCAAGACCCACGACTACAAAGACGATGACGACAA |
| | G (SEQ ID NO: 9) |
| Corresponding amino | MSVVCRSSCSLLLLPCLLLCVLGPSASHAGKLLVIPIDGSHWLS |
| acid sequence | MLGVIQQLQQKGHEVVVIAPEASIHIKEGSFYTMRKYPVPFQNE |
| | NVTAAFVELGRSVFDQDPFLLRVVKTYNKVKRDSSMLLSGCSHL |
| | LHNAEFMASLEQSHFDALLTDPFLPCGSIVAQYLSLPAVYFLNA |
| | LPCSLDLEATQCPAPLSYVPKSLSSNTDRMNFLQRVKNMIIALT |
| | ENFLCRVVYSPYGSLATEILQKEVTVKDLLSPASIWLMRNDFVK |
| | DYPRPIMPNMVFIGGINCLQKKALSQEFEAYVNASGEHGIVVFS |
| | LGSMVSEIPEKKAMEIAEALGRIPQTVLWRYTGTRPSNLAKNTI |
| | LVKWLPQNDLLGHPKARAFITHSGSHGIYEGICNGVPMVMMPLF |
| | GDQMDNAKRMETRGAGVTLNVLEMTADDLENALKTVINNKSYKE |
| | NIMRLSSLHKDRPIEPLDLAVFWVEYVMRHKGAPHLRPAAHDLT |
| | WYQYHSLDVIGFLLAIVLTVVFIVYKSCAYGCRKCFGGKGRVKK |
| | SHKSKTHDYKDDDDK (SEQ ID NO: 10) |

In some embodiments, the UGT1 or biologically active fragment thereof, encoded by the mRNA described herein, comprises a protein sequence with at least 70%, 75%, 80%, 85%, 90%, 91%, 92%, 93%, 94%, 95%, 96%, 97%, 98%, 99%, or 100% identity to at least one of SEQ ID NOS:4, 6, 8 or 10, or biologically active fragment thereof. The mRNA encoding a UGT1 or a biologically active fragment thereof, therefore, can comprise a nucleotide sequence with at least 70%, 75%, 80%, 85%, 90%, 91%, 92%, 93%, 94%, 95%, 96%, 97%, 98%, 99%, or 100% identity to a nucleotide sequence that encodes at least one of SEQ ID NOS: 4, 6, 8 or 10, or biologically active fragment thereof.

The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art.

Percent identity can be determined using a sequence alignment tool or program, including but not limited to (1) a BLAST 2.0 Basic BLAST homology search using blastp for amino acid searches and blastn for nucleic acid searches with standard default parameters, wherein the query sequence is filtered for low complexity regions by default; (2) a BLAST 2 alignment (using the parameters described below); (3) PSI BLAST with the standard default parameters (Position Specific Iterated BLAST; (4) and/or Clustal Omega. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches.

One of ordinary skill in the art will recognize that individual substitutions, deletions or additions to a nucleic acid, peptide, polypeptide or protein sequences that alter, add or delete a single amino acid or a small percentage of amino acids in the encoded sequence is a “conservatively modified variant.” Such variants can be useful, for example, to alter the physical properties of the peptide, e.g., to increase stability or efficacy of the peptide. Conservative substitution tables providing functionally similar amino acids are known to those of ordinary skill in the art. Such conservatively modified variants are in addition to and do not exclude polymorphic variants, interspecies homologs and alternate alleles. The following groups provide non limiting examples of amino acids that can be conservatively substituted for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The term “codon-optimized” refers to genes or coding regions of a nucleic acid molecule to be translated into a polypeptide sequence. Due to the degeneracy of the genetic code, there are typically more than one triplet codons that cade for a particular amino acid during translation. Some codons are more commonly used to encode a particular amino acid by particular organisms, and translation efficiency can be improved by changing the mRNA sequence in such a way as the desired codons are effectively used by the desired host translation machinery. This process, where the mRNA sequence is changed to reflect alternate codon usage to improve translation efficiency without affecting the sequence of the translated polypeptide, is referred to as “codon optimization.” One of skill in the art will recognize, that several algorithms are available to codon optimize an mRNA sequence in silico. In particular embodiments, the modified mRNA molecules are codon-optimized.

Codon usage bias refers to differences in the frequency of occurrence of synonymous codons in coding DNA (Hershberg, R. & Petrov, D., Annu. Rev. Genet., 42:287-99, 2008; Eyre-Walker, A. J. Mol. Evol., 33:442-9, 1991). A codon is a series of three nucleotides (triplets) that encodes a specific amino acid residue in a polypeptide chain or for the termination of translation (stop codons). There are 64 different codons (61 codons encoding for amino acids plus 3 stop codons) for only 20 different translated amino acids. The overabundance in the number of codons allows many amino acids to be encoded by more than one codon. Different organisms often show particular preferences for one of the several codons that encode the same amino acid. Codon preferences reflect a balance between mutational biases and natural selection for translational optimization. Optimal codon usage in fast-growing microorganisms, like Escherichia coli or Saccharomyces cerevisiae (baker's yeast), for example, reflects the composition of their respective genomic tRNA pool. Optimal codon usage may help to achieve faster translation rates and high accuracy. As a result of these factors, translational selection is expected to be stronger in highly expressed genes, as is indeed the case for the above-mentioned organisms.

In organisms that do not show high growing rates or that present small genomes, codon usage optimization is normally absent, and codon preferences are determined by the characteristic mutational biases seen in that particular genome. Examples of this are Homo sapiens (human) and Helicobacter pylori. Organisms that show an intermediate level of codon usage optimization include at least Drosophila melanogaster (fruit fly), Caenorhabditis elegans (nematode worm), Strongylocentrotus purpuratus (sea urchin) and Arabidopsis thaliana (thale cress).

The modRNA molecules described herein can comprise at least one codon substituted to create the corresponding biased codon specific to the mammal species for delivering such polynucleotide. One exemplary and non-limiting rationale for this substitution is to decrease host immunogenicity and/or to facilitate protein translation in such mammal species. Alternatively, an mRNA can comprise at least one codon substituted to a non-preferred codon in the host mammal species, as such substitutions allow one of skill in the art to attenuate translation speed and efficiency, e.g., to increase differentiation of the expressed protein and/or to add desired properties to the expressed protein or fragment thereof.

### RNA Formation and Modifications

As used herein, the term “nucleic acid” refers to polymeric biomolecules, e.g., genetic material (e.g., oligonucleotides or polynucleotides comprising DNA or RNA), which include any compound and/or substance that comprise a polymer of nucleotides. These polymers are polynucleotides. Nucleic acids described herein include, for example, RNA or stabilized RNA, e.g., modRNA, encoding a protein or enzyme.

The mRNAs described herein can be natural or recombinant, isolated or chemically synthesized. Such mRNAs can be, for example isolated from in vitro cell cultures or from organisms such as plants or animals in vivo. The mRNAs can be, for example, synthesized or produced in silico.

Described herein are compositions and methods for the manufacture and optimization of mRNA molecules, e.g., modRNAs, through modification of the architecture of mRNA molecules. The disclosure provides, for example, methods for increasing production of a UGT1 or a biologically active fragment thereof encoded by the mRNA molecules by altering mRNA sequence and/or structure.

The modRNA can comprise, for example, one or more chemical/structural modifications. Such modification(s) can, for example, reduce the innate immune response of a cell into which the mRNA molecule is introduced or any of plurality of other desired effects including, but not limited to: 1) improving the stability of the mRNA molecule; 2) improving the efficiency of protein production; 3) improving intracellular retention and/or the half-life of the mRNA molecules; and/or 4) improving viability of contacted cells. Exemplary modification methods and compositions can be seen in, for example, PCT publication Nos. WO2014081507 and WO2013151664, the entire contents of each of which are hereby incorporated by reference.

Provided herein is a modified mRNA molecule containing a translatable region and one, two or more than two different nucleoside modifications. Nucleoside modifications can include, for example, uniform substitution of a ribonucleoside throughout the modRNA, e.g., incorporation of a modified uracil, cytosine, adenine or guanine at every position where uracil, cytosine, adenine or guanine occurs in the mRNA sequence. Alternatively, modifications can occur at specific sequence positions, and thus the modRNA is discreetly modified. In some embodiments, the modRNA exhibits reduced degradation in a cell into which the mRNA is introduced, relative to a corresponding unmodified mRNA. Two or more linked nucleotides, for example, can be inserted, deleted, duplicated, inverted or randomized in the mRNA molecule without significant chemical modification to the mRNA. The chemical modifications can be located on the sugar moiety of an mRNA molecule described herein. The chemical modifications can be located on the phosphate backbone of the mRNA.

The modRNA molecule(s) described herein can be cyclized or concatemerized, to generate a translation competent molecule to assist interactions, for example, between poly-A binding proteins and 5′ end binding proteins. Cyclization or concatemerization can be achieved, for example, by 1) chemical, 2) enzymatic and/or 3) ribozyme catalyzed processes. The newly formed 5′-/3′-linkage can be intramolecular or intermolecular.

modRNA molecules can be, for example, linked using a functionalized linker molecule. A functionalized saccharide molecule, for example, can be chemically modified to contain multiple chemical reactive groups (SH—, NH₂—, N3, etc. . . . ) to react with the cognate moiety on a 3′-functionalized mRNA molecule (e.g., a 3′-maleimide ester, 3′-NHS-ester, alkynyl, etc.). The number of reactive groups on the modified saccharide can be controlled in a stoichiometric fashion to directly control the stoichiometric ratio of conjugated nucleic acid or mRNA.

The mRNA molecule(s) described herein can be conjugated to other polynucleotides, dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases, alkylating agents, phosphate, amino acids, PEG (e.g., PEG-40K), MPEG, [MPEG]₂, radiolabeled markers, enzymes, haptens (e.g., biotin), transport/absorption facilitators (e.g., aspirin, vitamin E, folic acid), synthetic ribonucleases, proteins (e.g., glycoproteins), peptides (e.g., molecules having a specific affinity for a co-ligand), antibodies (e.g., an antibody that binds to a specified cell type such as, for example, a cancer cell, endothelial cell, hepatocyte or bone cell), hormones and hormone receptors, non-peptidic species (such as lipids, lectins, carbohydrates, vitamins, and cofactors), or a drug. Conjugation may result in increased stability and/or half-life and may be particularly useful in targeting the mRNA molecule of the instant disclosure to specific sites in the cell, tissue or organism.

An mRNA molecule described herein can be, for example bi-functional, which means the mRNA molecule has or is capable of two functions, or multi-functional. The multiple functionalities, structural or chemical, can be encoded by the mRNA (e.g., the function may not manifest until the encoded product is translated) or may be a property of the mRNA itself. Similarly, bi-functional mRNA molecules may comprise a function that is covalently or electrostatically associated with the mRNA. Multiple functions may be provided in the context of a complex of a modified RNA and another molecule.

The mRNA molecule can be purified after isolating from a cell, a tissue, or an organism or chemically synthesized. The purification process may include, for example, clean-up, quality assurance, and quality control. Purification may be performed by methods known in the arts such as, for example, chromatographic methods, e.g., using, for example, AGENCOUIRT® beads (Beckman Coulter Genomics, Danvers, Mass.), poly-T beads, LNA™ oligo-T capture probes (EXIQON® Inc, Vedbaek, Denmark) or HPLC-based purification methods such as, for example, strong anion exchange HPLC, weak anion exchange HPLC, reverse phase HPLC (RP-HPLC), and hydrophobic interaction HPLC (HIC-HPLC). A purified polynucleotide (e.g., mRNA) is present in a form or setting different from that in which it is found in nature or a form or setting different from that in which it existed prior to subjecting it to a treatment or purification method.

A quality assurance and/or quality control check may be conducted using methods such as, but are not limited to, gel electrophoresis, UV absorbance, or analytical HPLC. In another embodiment, the mRNA molecule may be sequenced by methods including, but not limited to, reverse-transcriptase-PCR.

In one embodiment, the mRNA molecule is quantified using methods such as, for example, ultraviolet visible spectroscopy (UV/Vis). The mRNA molecule can be analyzed to determine if the mRNA is of proper size or if degradation has occurred. Degradation of the mRNA can be checked by methods such as, for example, agarose gel electrophoresis, HPLC based purification methods (e.g., strong anion exchange HPLC, weak anion exchange HPLC, reverse phase HPLC (RP-HPLC), and hydrophobic interaction HPLC (HIC-HPLC)), liquid chromatography/mass spectrometry (LCMS), capillary electrophoresis (CE) and capillary gel electrophoresis (CGE).

The described mRNA can comprise at least one structural or chemical modification. The nucleoside that is modified in the mRNA, for example, can be a uridine (U), a cytidine (C), an adenine (A), or guanine (G). The modified nucleoside can be, for example, m⁵C (5-methylcytidine), m⁶A (N6-methyladenosine), s²U (2-thiouridien), ψ (pseudouridine) or Um (2-O-methyluridine). Some exemplary chemical modifications of nucleosides in the mRNA molecule further include, for example, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyl adenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, and N²,N²-dimethyl-6-thio guanosine. In another embodiment, the modifications are independently selected from the group consisting of 5-methylcytosine, pseudouridine and 1-methylpseudouridine.

In some embodiments, the modified nucleobase in the mRNA molecule is a modified uracil including, for example, pseudouridine (ψ), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmo⁵U), uridine 5-oxyacetic acid methyl ester (mcmo⁵U), 5-carboxymethyl uridine (cm⁵U), 1-carboxymethyl pseudouridine, 5-carboxyhydroxymethyl uridine (chm⁵U), 5-carboxyhydroxymethyl uridine methyl ester (mchm⁵U), 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm⁵s2U), 5-methylaminomethyl-2-seleno uridine (mnm⁵se²U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (τcm⁵U), 1-taurinomethyl pseudouridine, 5-taurinomethyl-2-thio uridine (τm⁵s2U), 1-taurinomethyl-4-thio pseudouridine, 5-methyl uridine (m⁵U, e.g., having the nucleobase deoxythymine), 1-methyl pseudouridine (m¹ψ) 5-methyl-2-thio uridine (m⁵s2U), 1-methyl-4-thio pseudouridine (m¹s⁴ψ), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), .alpha-thio uridine, 2′-O-methyl uridine (Um), 5,2′-O-dimethyl uridine (m⁵Um), 2′-O-methyl pseudouridine (ψm), 2-thio-2′-O-methyl uridine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inm⁵Um), 1-thio uridine, deoxythymidine, 2′-F-ara uridine, 2′-F uridine, 2′-OH-ara uridine, 5-(2-carbomethoxyvinyl) uridine, and 5-[3-(1-E-propenylamino) uridine.

In some embodiments, the modified nucleobase is a modified cytosine including, for example, 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm⁵C), 1-methyl pseudoisocytidine, pyrrolo-cytidine, pyrrolo-pseudoisocytidine, 2-thio cytidine (s2C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, lysidine (k²C), alpha-thio cytidine, 2′-O-methyl cytidine (Cm), 5,2′-O-dimethyl cytidine (m⁵Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴₂Cm), 1-thio cytidine, 2′-F-ara cytidine, 2′-F cytidine, and 2′-OH-ara cytidine.

In some embodiments, the modified nucleobase is a modified adenine including, for example, 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms²m⁶A), N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t⁶A), 2-methylthio-N⁶-threonylcarbamoyl adenosine (ms²g⁶A), N⁶,N⁶-dimethyl adenosine (m⁶₂A), N⁶-hydroxynorvalylcarbamoyl adenosine (hn⁶A), 2-methylthio-N⁶-hydroxynorvalylcarbamoyl adenosine (ms² hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am), N⁶,N⁶,2′-O-trimethyl adenosine (m⁶₂Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, the modified nucleobase is a modified guanine including, for example, inosine (I), 1-methyl inosine (m¹I), wyosine (imG), methylwyosine (mimG), 4-demethyl wyosine (imG-14), isowyosine (imG2), wybutosine (yW), peroxywybutosine (o₂yW), hydroxywybutosine (OHyW), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archaeosine (G⁺), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosine (m²₂G), N^{2,7}-dimethyl guanosine (m^{2,7}G), N², N^{2,7}-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N^{2,7}-dimethyl-2′-O-methyl guanosine (m^{2,7}Gm), 2′-O-methyl inosine (Im), 1,2′-O-dimethyl inosine (m¹Im), 2′-O-ribosyl guanosine (phosphate) (Gr(p)), 1-thio guanosine, O⁶-methyl guanosine, 2′-F-ara guanosine, and 2′-F guanosine.

The nucleobase of the nucleotide can be independently selected from a purine, a pyrimidine, a purine or pyrimidine analog. For example, the nucleobase can each be independently selected from adenine, cytosine, guanine, uracil or hypoxanthine. The nucleobase can also include, for example, naturally occurring and synthetic derivatives of a base, including, but not limited to, pyrazolo[3,4-d]pyrimidines, 5-methylcytosine (5-me-C), 5-hydroxymethyl cytosine, xanthine, hypoxanthine, 2-amino adenine, 6-methyl and other alkyl derivatives of adenine and guanine, 2-propyl and other alkyl derivatives of adenine and guanine, 2-thio uracil, 2-thio thymine and 2-thio cytosine, 5-propynyl uracil and cytosine, 6-azo uracil, cytosine and thymine, pseudouracil, 4-thio uracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methyl guanine and 7-methyl adenine, 8-aza guanine and 8-aza adenine, deaza guanine, 7-deaza guanine, 3-deaza guanine, deaza adenine, 7-deaza adenine, 3-deaza adenine, pyrazolo[3,4-d]pyrimidine, imidazo[1,5-a]1,3,5 triazinones, 9-deaza purines, imidazo[4,5-d]pyrazines, thiazolo[4,5-d]pyrimidines, pyrazine-2-ones, 1,2,4-triazine, pyridazine; and 1,3,5-triazine. When the nucleotides are depicted using the shorthand A, G, C, T or U, each letter refers to the representative base and/or derivatives thereof, e.g., A includes adenine or adenine analogs, e.g., 7-deaza adenine).

Other modifications include, for example, those in U.S. Pat. No. 8,835,108; U.S. Patent Application Publication No. 20130156849; Tavernier, G. et al., J. Control. Release, 150:238-47, 2011; Anderson, B. et al., Nucleic Acids Res., 39:9329-38, 2011; Kormann, M. et al., Nat. Biotechnol., 29:154-7, 2011; Karikó, K. et al., Mol. Ther., 16:1833-40, 2008; Karikó, K. et al., Immunity, 23:165-75, 2005; and Warren, L. et al., Cell Stem Cell, 7:618-30, 2010; the entire contents of each of which is incorporated herein by reference.

### Compositions

The mRNA described herein can be delivered into a host, such as a mammal (e.g., a human), to express a protein of interest (e.g., a UGT1 or biologically active fragment thereof). The mRNA can comprise an exon of the protein of interest for in vivo expression. Optionally, the mRNA can have at least one of the introns of the protein of interest or another protein to facilitate gene expression. For the encoded UGT1 or biologically active fragment(s) thereof, different subunit polypeptides or domains of the same or different subunit polypeptides can be expressed from a single mRNA molecule or from two different mRNA molecules (e.g., each chain expressing a different subunit). In latter situation these two mRNA molecules can be co-delivered into the host for in vivo expression. Optionally, the one or two mRNA molecule can be delivered in conjunction with a polypeptide or protein, or an mRNA encoding such polypeptide or protein, which is capable of facilitating protein expression of the UGT1 or biologically active fragments thereof (e.g., co-expression of one or more biologically active fragments).

### Delivery

When formulated in a nanoparticle for delivery, modified mRNA show increased nuclease tolerance and is more effectively taken up by tumor cells after systemic administration (Wang, Y. et al., Mol. Ther., 21:358-67, 2013; the content of which is incorporated by reference herein in its entirety). mRNA can be delivered, for example, by multiple methods to the host organism (PCT publication Nos: WO2013185069, WO2012075040 and WO2011068810, the entire contents of each of which is herein incorporated by reference).

Lipid carrier vehicles can be used to facilitate the delivery of nucleic acids to target cells. Lipid carrier vehicles (e.g., liposomes and lipid-derived nanoparticles (LNPs), such as, for example, the MC3 LNP (Arbutus Biopharma)) are generally useful in a variety of applications in research, industry, and medicine, particularly for their use as transfer vehicles of diagnostic or therapeutic compounds in vivo (Lasic, D., Trends Biotechnol., 16:3-7-21, 1998; Drummond, D. et al., Pharmacol. Rev., 51:691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains.

The liposomal transfer vehicles are prepared to contain the desired nucleic acids for the protein of interest. The process of incorporation of a desired entity (e.g., a nucleic acid such as, for example, an mRNA) into a liposome is referred to as “loading” (Lasic, D. et al., FEBS Lett., 312:255-8, 1992). The liposome-incorporated nucleic acids can be completely or be partially located in the interior space of the liposome, within the bilayer membrane of the liposome, or associated with the exterior surface of the liposome membrane. The incorporation of a nucleic acid into liposomes is referred to herein as “encapsulation,” wherein the nucleic acid is entirely contained within the interior space of the liposome. The purpose of incorporating an mRNA into a transfer vehicle, such as a liposome, is often to protect the nucleic acid from an environment that may contain enzymes or chemicals that degrade nucleic acids and/or systems or receptors that cause the rapid excretion of the nucleic acids. Accordingly, the selected transfer vehicle is capable of enhancing the stability of the mRNA contained therein. The liposome allows the encapsulated mRNA to reach a desired target cell.

As used herein, the term “target cell” refers to a cell or tissue to which a composition described herein is to be directed or targeted. In some embodiments, the target cells are deficient in a protein or enzyme of interest. For example, where it is desired to deliver a nucleic acid to a hepatocyte, the hepatocyte represents the target cell. In some embodiments, the nucleic acids and compositions specifically transfect the target cells (i.e., they do not transfect non-target cells). The compositions and methods can be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions described herein can be administered and dosed in accordance with current medical practice, taking into account, for example, the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient expression of the antibody or fragment in the target cell.

The route of delivery used in the methods of the disclosure allows for noninvasive, self-administration of the therapeutic compositions of mRNA described herein. The methods involve intratracheal or pulmonary administration by aerosolization, nebulization, or instillation of compositions comprising the mRNA in a suitable transfection or lipid carrier vehicles as described herein.

Following administration of the composition to the subject, the protein of interest, e.g., UGT1 or biologically active fragment(s) thereof encoded by the mRNA, is detectable in the target tissues for at least about one to about seven days or longer following administration of the composition to the subject. The amount of expressed protein or protein fragment necessary to achieve a therapeutic effect varies depending on the condition being treated and the condition of the patient. The expressed UGT1 or fragment(s), for example, is detectable in the target tissues at a concentration of at least 0.025-1.5 μg/mL (e.g., at least 0.050 μg/mL, at least 0.075 μg/mL, at least 0.1 μg/mL, at least 0.2 μg/mL, at least 0.3 μg/mL, at least 0.4 μg/mL, at least 0.5 μg/mL, at least 0.6 μg/mL, at least 0.7 μg/mL, at least 0.8 μg/mL, at least 0.9 μg/mL, at least 1.0 μg/mL, at least 1.1 μg/mL, at least 1.2 μg/mL, at least 1.3 μg/mL, at least 1.4 μg/mL, or at least 1.5 μg/mL), or at a higher concentration, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40 or 45 days or longer following administration of the composition to the subject.

### Pharmaceutical Compositions and Formulations

The mRNA compositions described herein can be formulated as a pharmaceutical solution, e.g., for administration to a subject for the treatment or prevention of a disease or disorder associated with UGT1 deficiency, e.g., CN1. The pharmaceutical compositions can include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (Berge. S. et al., J. Pharm. Sci., 66:1-19, 1977).

The compositions can be formulated according to methods in the art (Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). A composition can be formulated, for example, as a buffered solution at a suitable concentration and suitable for storage at 2-8 C (e.g., 4 C). In some embodiments, a composition can be formulated for storage at a temperature below 0 C (e.g., −20 C or −80 C). In some embodiments, the composition can be formulated for storage for up to two years (e.g., one month, two months, three months, four months, five months, six months, seven months, eight months, nine months, 10 months, 11 months, 1 year, 1½ years or 2 years). Thus, in some embodiments, the compositions described herein are stable in storage for at least one year at 2-8 C (e.g., 4 C).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing an mRNA molecule intended for systemic or local delivery can be in the form of injectable or infusible solutions. Accordingly, the compositions can be formulated for administration by a parenteral mode (e.g., intravenous, subcutaneous, intraperitoneal or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

The compositions can be formulated as a solution, microemulsion, dispersion, liposome or other ordered structure suitable for stable storage at high concentration. Sterile injectable solutions can be prepared by incorporating a composition described herein in the required amount in an appropriate solvent with one or a combination of ingredients enumerated above, as required or otherwise desirable, followed by filter sterilization. Dispersions are generally prepared by incorporating a composition into a sterile vehicle that contains a basic dispersion medium and other ingredients from those enumerated above. In the case of sterile powders for the preparation of sterile injectable solutions, methods for preparation include vacuum drying and freeze-drying that yield a powder of a composition plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts and gelatin.

The mRNA compositions described herein can also be formulated in liposome compositions prepared by methods known in the art (e.g., Eppstein, D. et al., Proc. Natl. Acad. Sci. USA, 82:3688-92, 1985; Hwang, K. et al., Proc. Natl. Acad. Sci. USA, 77:4030-4, 1980; and U.S. Pat. Nos. 4,485,045; 4,544,545 and U.S. Pat. No. 5,013,556; the entire contents of each of which is incorporated by reference herein).

Compositions can be formulated with a carrier, for example, which protects the formulated mRNA against rapid release, such as a controlled release formulation, including implants and microencapsulated delivery systems. Biodegradable, biocompatible polymers, for example, can be used (e.g., ethylene vinyl acetate, polyanhydrides, polyglycolic acid, collagen, polyorthoesters and polylactic acid). Many methods for the preparation of such formulations are known in the art (e.g., J. R. Robinson (1978) “Sustained and Controlled Release Drug Delivery Systems,” Marcel Dekker, Inc., New York).

Compositions can be formulated for delivery to the eye. As used herein, the term “eye” refers to any and all anatomical tissues and structures associated with an eye.

In some embodiments, compositions can be administered locally, for example, by way of topical application or intravitreal injection. For example, in some embodiments, the compositions can be formulated for administration by way of an eye drop.

The therapeutic preparation for treating the eye can contain one or more active agents in a concentration from about 0.01 to about 1% by weight, preferably from about 0.05 to about 0.5% in a pharmaceutically acceptable solution, suspension or ointment. The preparation can be, for example, in the form of a sterile aqueous solution containing, e.g., additional ingredients such as, but are not limited to, preservatives, buffers, tonicity agents, antioxidants and stabilizers, nonionic wetting or clarifying agents and viscosity-increasing agents.

Suitable preservatives for use in such a solution include, for example, benzalkonium chloride, benzethonium chloride, chlorobutanol, thimerosal and the like. Suitable buffers include, e.g., boric acid, sodium and potassium bicarbonate, sodium and potassium borates, sodium and potassium carbonate, sodium acetate, and sodium biphosphate, in amounts sufficient to maintain the pH at between about pH 6 and about pH 8, and preferably, between pH 7 and pH 7.5. Suitable tonicity agents include, for example, dextran 40, dextran 70, dextrose, glycerin, potassium chloride, propylene glycol and sodium chloride.

Suitable antioxidants and stabilizers include, for example, sodium bisulfite, sodium metabisulfite, sodium thiosulfite and thiourea. Suitable wetting and clarifying agents include, for example, polysorbate 80, polysorbate 20, poloxamer 282 and tyloxapol. Suitable viscosity-increasing agents include, for example, dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone and carboxymethylcellulose.

As described above, relatively high concentration (mRNA) compositions can be made. For example, the compositions can be formulated at an mRNA concentration between about 10 mg/mL to about 100 mg/mL (e.g., between about 9 mg/mL and about 90 mg/mL; between about 9 mg/mL and about 50 mg/mL; between about 10 mg/mL and about 50 mg/mL; between about 15 mg/mL and about 50 mg/mL; between about 15 mg/mL and about 110 mg/mL; between about 15 mg/mL and about 100 mg/mL; between about 20 mg/mL and about 100 mg/mL; between about 20 mg/mL and about 80 mg/mL; between about 25 mg/mL and about 100 mg/mL; between about 25 mg/mL and about 85 mg/mL; between about 20 mg/mL and about 50 mg/mL; between about 25 mg/mL and about 50 mg/mL; between about 30 mg/mL and about 100 mg/mL; between about 30 mg/mL and about 50 mg/mL; between about 40 mg/mL and about 100 mg/mL; or between about 50 mg/mL and about 100 mg/mL). In some embodiments, compositions can be formulated at a concentration of greater than 5 mg/mL and less than 50 mg/mL. Methods for formulating a protein in an aqueous solution are known in the art, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (2005), “Therapeutic peptides and proteins: formulation, processing, and delivery systems, Second Edition” CRC Press.

In some embodiments, the aqueous solution has a neutral pH, e.g., a pH between, e.g., 6.5 and 8 (e.g., between and inclusive of 7 and 8). In some embodiments, the aqueous solution has a pH of about 6.6, 6.7, 6.8, 6.9, 7, 7.1, 7.2, 7.3, 7.4, 7.5, 7.6, 7.7, 7.8, 7.9 or 8.0. In some embodiments, the aqueous solution has a pH of greater than (or equal to) 6 (e.g., greater than or equal to 6.1, 6.2, 6.3, 6.4, 6.5, 6.6, 6.7, 6.8, 6.9, 7, 7.1, 7.2, 7.3, 7.4, 7.5, 7.6, 7.7, 7.8 or 7.9), but less than pH 8.

In some embodiments, compositions can be formulated with one or more additional therapeutic agents, e.g., additional therapies for treating or preventing a disease or disorder described herein, e.g., UGT1-deficiency-associated disease or disorder in a subject. When compositions are to be used in combination with a second active agent, the compositions can be co-formulated with the second agent or the compositions can be formulated separately from the second agent formulation. The respective pharmaceutical compositions can be mixed, for example, just prior to administration, and administered together or can be administered separately, e.g., at the same or different times.

### EXAMPLE

The Examples that follow are illustrative of specific embodiments of the invention, and various uses thereof. They are set forth for explanatory purposes only, and should not be construed as limiting the scope of the invention in any way.

Cell lines

HeLa and Clone 9 were purchased from ATCC (Manassas, Va.) and Sigma (St. Louis, Mo.) respectively and maintained according to provider's instructions. GM09551 and GM09705 CN1 patient-derived fibroblasts were purchased from Coriell Institute for Medical Research (Camden, N.J.). Gunn rat primary hepatocytes and Cynomolgus primary hepatocytes were purchased from Triangle Research Laboratories (Durham, N.C.) and In Vitro ADMET Laboratories (Columbia, Md.), and maintained according to provider's instructions.

Cultured cells lines have little or no expression of UGT1A1.

The Gunn rat is used as a model for Crigler-Najjar type 1 disease, as this animal model presents a single nucleotide polymorphism that leads to generation of a premature stop codon with undetectable levels of UGT1A1 protein and complete lack of activity.

### Cell Culture Media, Reagents and Buffers

HeLa, Clone 9 and CN1 patient-derived fibroblasts were maintained in Eagle's MEM (Corning, Manassas, Va.) supplemented with 10% heat inactivated fetal bovine serum (Tissue Culture Biologicals, Long Beach, Calif.) and 2 mM L-glutamine (Corning, Manassas, Va.).

Gunn rat primary hepatocytes were plated in animal hepatocyte plating media (Triangle Research Labs, Durham, N.C.) and maintained in hepatocyte maintenance media (Triangle Research Labs, Durham, N.C.). Cynomolgus primary hepatocytes were plated with UPCM™ IVAL Universal Primary Cell Plating Medium and maintained in HQM™ Hepatocyte Incubation Media (Columbia, Md.).

Chemical reagents used for microsomal isolation were purchased from Sigma (St. Louis, Mo.)

Antibodies (Western Blot and CE): Human UGT1A1, Rat UGT1A1, β-actin, Calnexin, DDDDK (FLAG), ERP72 and GADPH

Antibodies used include Rabbit monoclonal [EPR9592] anti UGT1A1 (Cat No. AB170858, Abcam, Cambridge, Mass.), mouse monoclonal anti-UGT1A1 (Cat No. mAB6490, R&D Systems, Minneapolis, Minn.), goat polyclonal anti-UGT1A1 (Cat No. sc-27419, Santa Cruz Biotechnology, Dallas, Tex.), Mouse anti-β-actin (Cat No. 3700S, Cell Signaling Technologies, Danvers, Mass.), rabbit polyclonal anti-calnexin (Cat No. AB22595, Abcam, Cambridge, Mass.), goat polyclonal anti-DDDDK (Cat No. AB1257 Abcam, Cambridge, Mass.) and mouse monoclonal anti-GAPDH (Cat No. AB125247 Abcam, Cambridge, Mass.).

### Immuno Blot

Human and rat UGT1A1 protein expression was measured either by standard chemilluminscence, by infrared, fluorescence-based Western blot methods or by capillary electrophoresis (CE). Immunoblot images were acquired using FluorChemo R system (ProteinSimple, San Jose, Calif.) or Odyssey CLx instrument (Li-Cor, Lincoln, Nebr.).

### UGT1A1 Enzyme Assay Method

UGT1A1 enzyme activity was measured using an HPLC assay (Nguyen, N. et al., J. Biol. Chem., 2837901-11, 2008).

### Liver Microsome Preparation

Liver from each rat was homogenized in 2 mL of ice cold 1× PBS supplemented with a protease inhibitor cocktail using IKA tissue homogenizer at 13,500 rpm while on ice. The tissue homogenate was first centrifuged at 12,331×g for 20 min at 4 C, and this resulting supernatant was centrifuged at 107,340×g for 60 min at 4 C. The pellet was suspended in microsome buffer (2.62 mM monobasic potassium phosphate, 1.38 mM dibasic potassium phosphate, 0.5 mM dithiothreitol and 0.2% glycerol), and protein concentration was determined by the Bradford method. Microsome preparations were used for protein expression detection (immunoblot or capillarity electrophoresis) and UGT1A1 enzyme activity analyses.

UGT1A1 Level in Immortalized Cells after Transfection with UGT1A1 modRNA

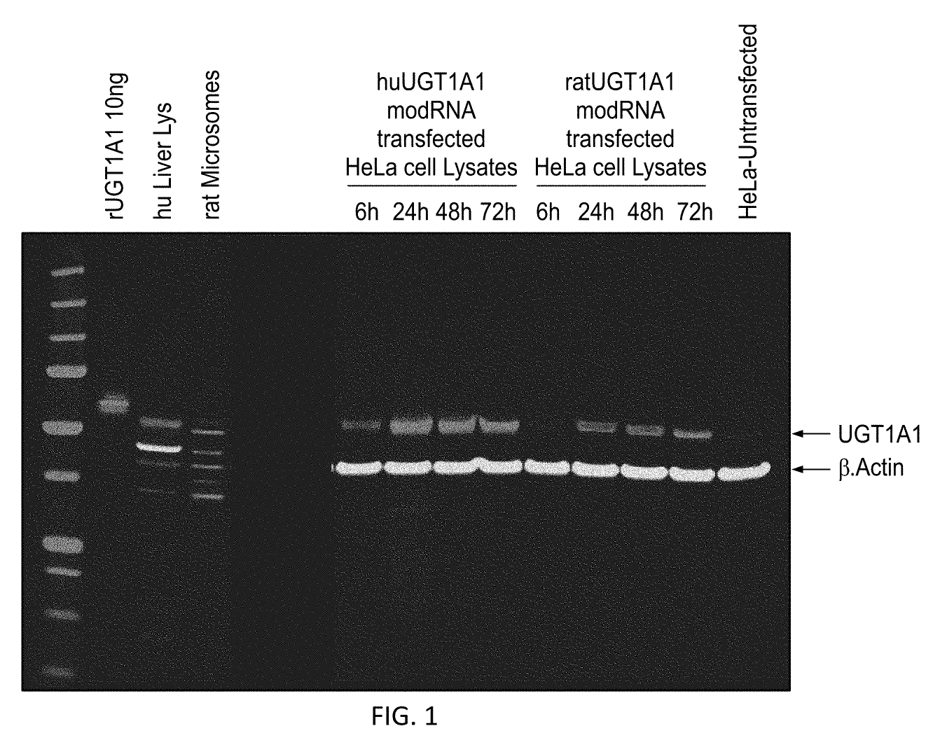

Immortalized human cell line (HeLa) expressed UGT1A1 with transfection of UGT1A1 modRNAs (human UGT1A1 and rat UGT1A1 modified with replacement of uridine with N1-methyl pseudouridine). An immunoreactive 52-kDa specific band corresponding to UGT1A1 was detected in protein extracts from UGT1A1 modRNA transduced cells and absent in non-transfected cells (FIG. 1).

This example also shows sustained UGT1A1 expression for three days in culture post-transfection with UGT1A1 modRNA (FIG. 1).

Compared Expression from Human and ratUGT1A and their Flag-Tagged Variant modRNA in Gunn Rat Primary Hepatocytes

Both human UGT1A1 and rat UGT1A1 modRNA (N1-methyl pseudouridine) and their FLAG-tagged variant modRNA expressed UGT1A1, and the newly synthetized proteins were functional in Gunn rat primary hepatocytes.

modRNA encoding C-terminal FLAG-tagged hUGT1A1 or rUGT1A1 were synthesized to facilitate distinction of modRNA-expressed proteins from endogenous UGT1A1 if experiments were to be conducted in wild-type animals where endogenous UGT1A1 is present.

Gunn rat primary hepatocytes (4.5×10⁵ cells) were transfected with modRNA encoding untagged hUGT1A1, hUGT1A1 with C-terminal FLAG, untagged rUGT1A1 or rUGT1A1 with C-terminal FLAG (2 μg modRNA).

After 24 hours, cells were harvested, and cell lysates were prepared for immunoblot analysis of hUGT1A1, rUGT1A1, FLAG and β-actin. UGT1A1 enzymatic activity was also measured.

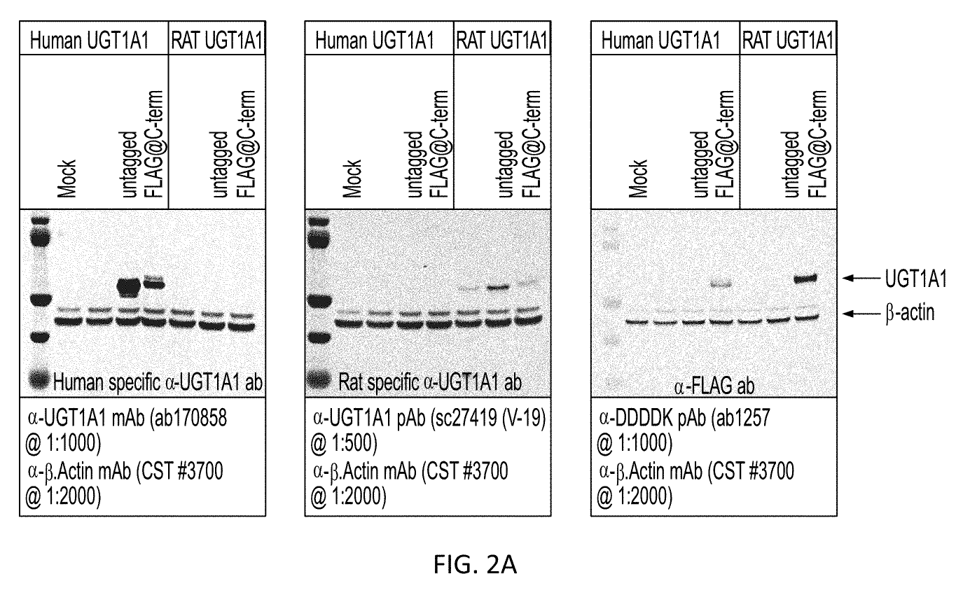

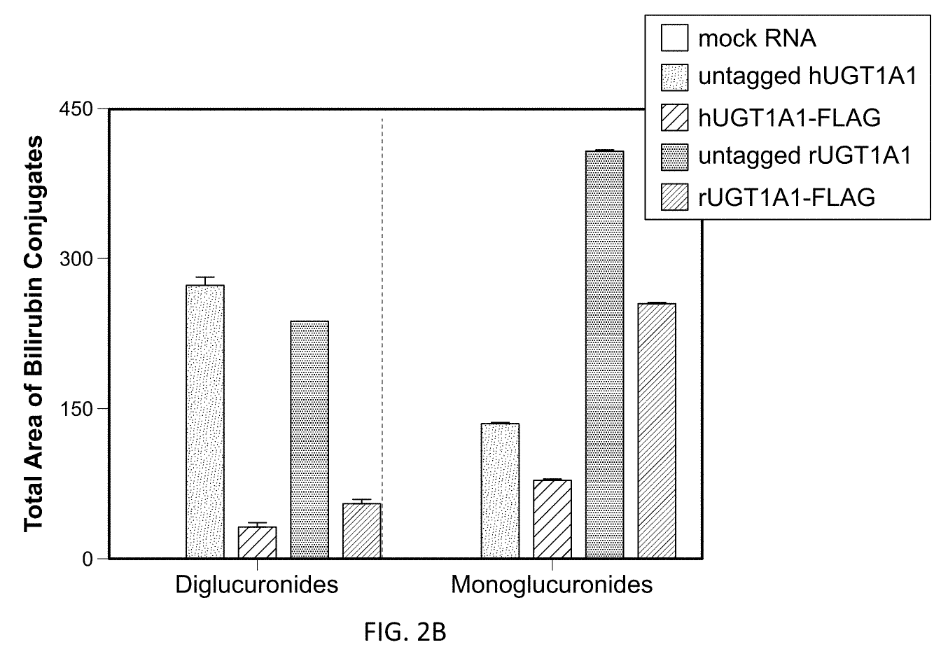

UGT1A1 level was detected with transfection of all four modRNAs and absent in non-transfected hepatocytes (FIG. 2A).

A reduced UGT1A1 protein level was observed for the C-terminally tagged variants. The presence of the FLAG on the C-terminus might be compromising the protein stability since UGTs are anchored to the endoplasmic reticulum (ER) membrane by a single C-terminal transmembrane helix (Laakkonen, L & Finel, M., Mol. Pharmacol., 77:931-9, 2010; Ciotti, M. et al., Biochemistry, 37:11018-25, 1998; Ouzzine, M. et al., FEBS Lett., 454:187-91, 1999).

In agreement with the lower levels of UGT1A1 protein, UGT1A1 enzyme activity was lower for C-terminally tagged variants compared to the untagged version independent of the species. The human UGT1A1 enzyme showed a lower level of monoglucuronides compared to the rat UGT1A1, however diglucuronides levels were similar (FIG. 2B).

UGT1A1 in CN1 Patient Fibroblast after Transfection with UGT1A1 modRNA

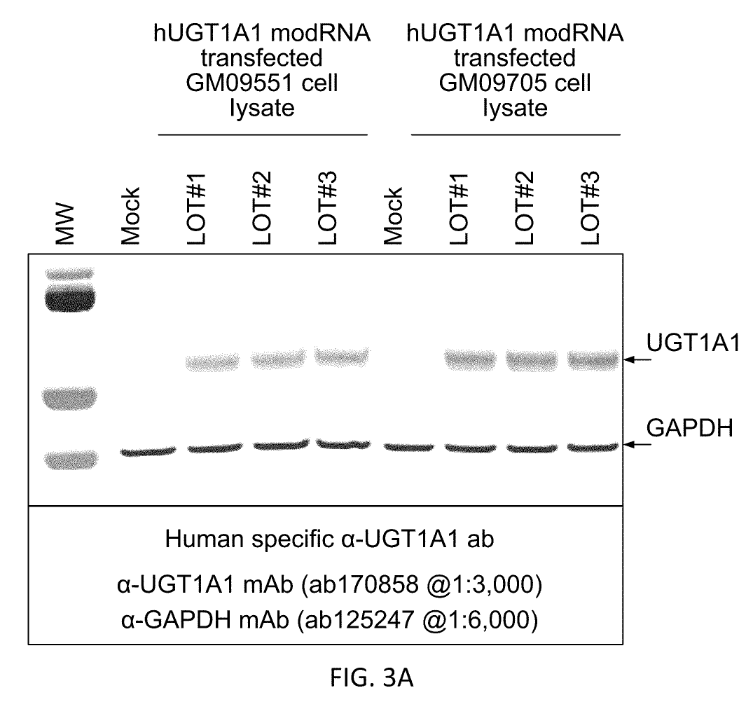

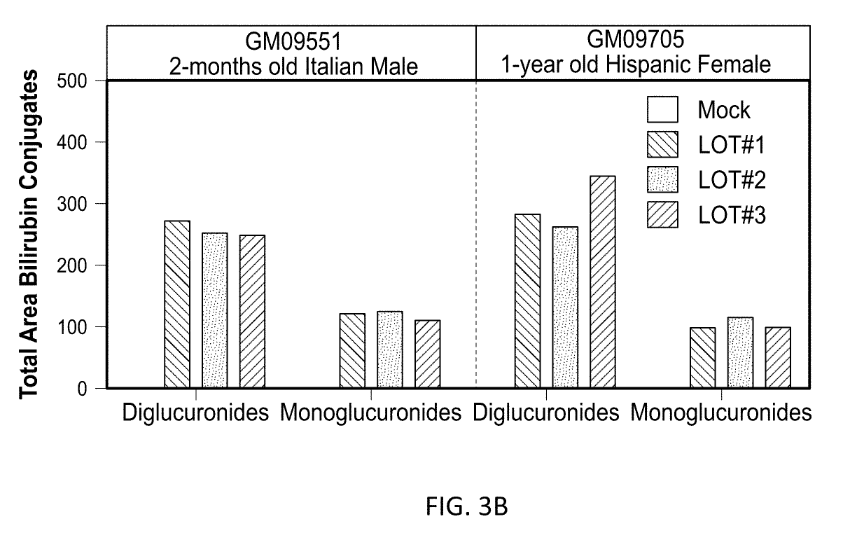

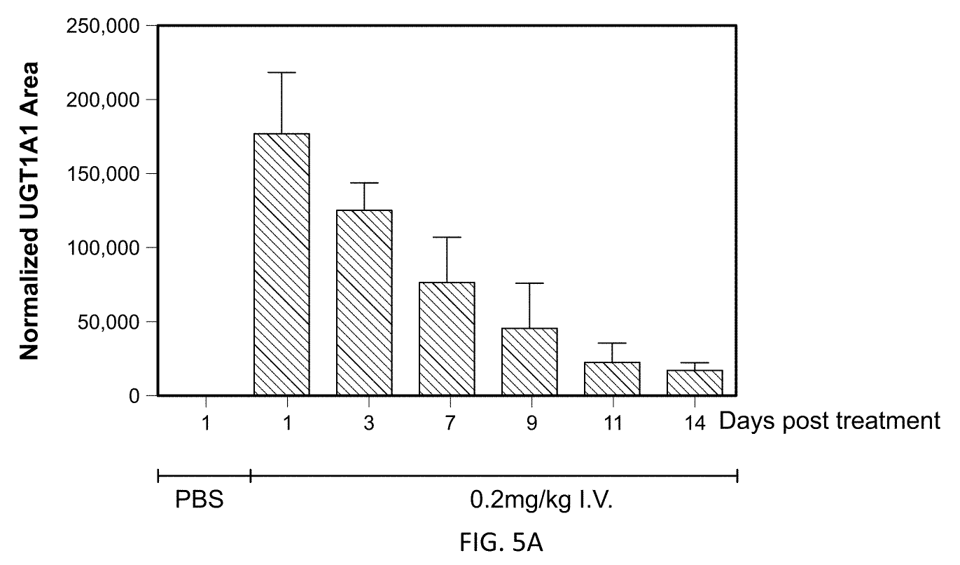

Fibroblast derived from two CN1 patients of different origins expressed UGT1A1 after transfection of human UGT1A1 modRNA. An immunoreactive 52-kDa specific band corresponding to UGT1A1 was detected in protein extracts from UGT1A1 modRNA transduced cells and absent in mock-transfected cells (FIG. 3A).

CN1 patient fibroblasts were transfected with three different modRNA lots encoding the hUGT1A1 (2 μg modRNA). After 24 hours, cells were harvested and cell lysates were prepared for immunoblot analysis of hUGT1A1 and GAPDH, and UGT1A1 enzymatic activity was measured. p All three modRNA lots tested showed similar UGT1A1 expression levels demonstrating consistency of the three lots and more importantly the ability of UGT1A1 modRNA to express protein in a human cell line, especially in CN1 patient-derived cells (FIG. 3A).

UGT1A1 enzyme activity correlated with hUGT1A1 expression, whereas similar levels of bilirubin conjugates were detected for all three modRNA tested in both CN1 patient-derived fibroblasts. No mono- or di-glucuronides were observed with mock control (FIG. 3B).

Localization of UGT1A1 Expressed from modRNA

Human UGT1A1 expressed from modRNA is correctly localized to the ER in both in vitro and in vivo transfected cells.

UGT1A1 is the most important enzyme from phase II metabolism. In vertebrates the conjugation step occurs within the ER where UGT1A1, an ER protein located at the luminal side and anchored to the membrane, transfers the glucuronic acid moiety to bilirubin. To study whether hUGT1A1 protein expressed from modRNA is correctly localized to their site of function, a localization study using immunofluorescence was performed.

Clone 9, a rat liver cell line with remarkably low endogenous UGT1A1, was selected as the cell model for this study.

In non-transfected control cells, ER stained with calnexin appeared as a net. No UGT1A1 signal was detected in non-transfected cells.

In cells transfected with human UGT1A1 modRNA, co-localization of UGT1A1 signal (red) with the Calnexin signal (green) was shown by the merged image (yellow). The immunofluorescent images demonstrate that hUGT1A1 proteins expressed from modRNA are properly targeting the ER (FIG. 4).

In Vivo 21-day Time Course in Gunn Rat Model Post Single Injection of hUGT1A1 modRNA

hUGT1A1 protein expressed from modRNA was detected in liver microsomes up to 14 days post single intravenous (i.v.) injection of Gunn rats dosed at 0.2 mg/kg with hUGT1A1 modRNA.

Gunn rat animals at 4-5 weeks of age were treated with hUGT1A1 modRNA at 3 different concentrations. A total of 95 Gunn rats distributed in groups of 5 animals per time point received bolus dosing of 0.1, 0.2 or 0.5 mg/kg at T₀ by the tail vein. Animals were euthanized at 1, 3, 7, 9, 11, 14 and 21 days after injection, and liver microsomes were prepared immediately after sacrifice. PBS-treated animals (wild-type and heterozygous) were used as negative controls and euthanized 1 day after i.v. injection. Human UGT1A1 level was detected by capillary electrophoresis (CE) and normalized by ERP72 area signal, which was used as protein loading control for animals in the 0.2 mg/kg group.

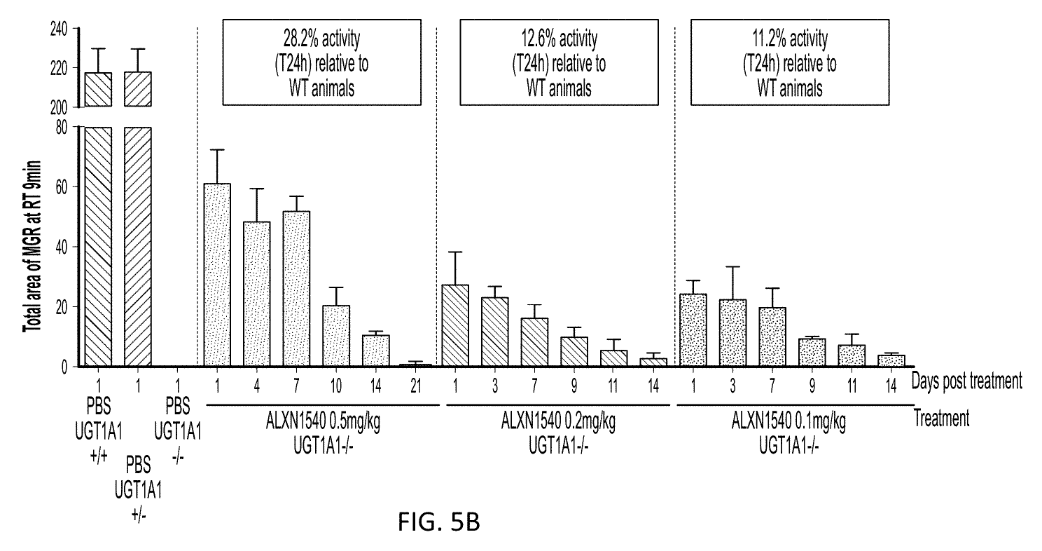

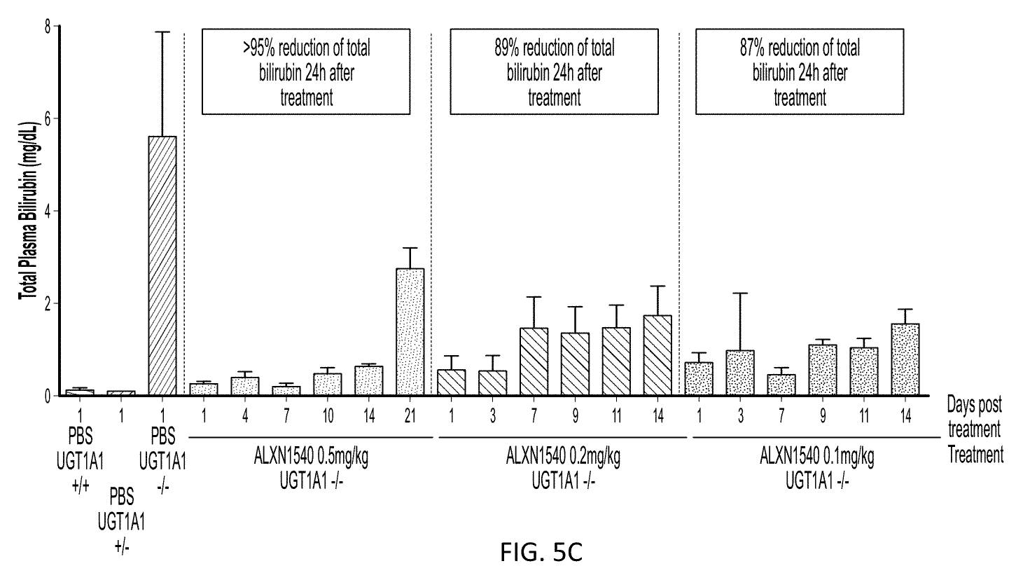

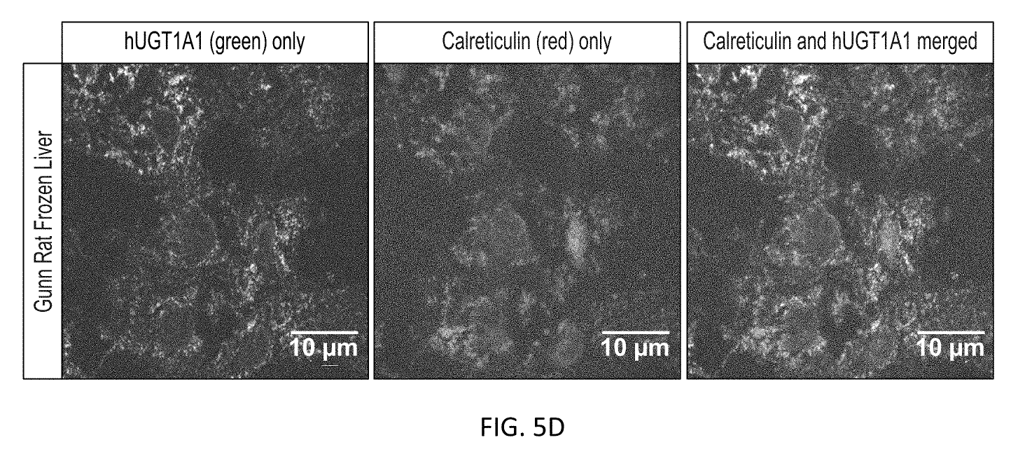

The highest UGT1A1 level was detected at 1 day after injection of 0.2 mg/kg and gradually went down. Remarkably, UGT1A1 can still be detected 14 days after single injection—demonstrating a longer half-life for human UGT1A1 than the rat UGT1A1 (10 hour half-life (FIG. 5A); Emi, Y. et al., Arch. Biochem. Biophys., 405:163-9, 2002)).

UGT1A1 enzyme activity corresponds to hUGT1A1 levels. The highest monoglucuronides levels were detected one day after modRNA injection. After single treatment with 0.1, 0.2 and 0.5 mg/kg, UGT1A1 expressed from modRNA restored 11.2, 12.6 and 28.2% of monoglucuronides levels obtained from liver microsomes of

### PBS-Treated Animals (WT or Heterozygous) Demonstrating a Dose-Dependent Effect (FIG. 5B).

Gunn rats have been used as model for Crigler-Najjar type 1 disorder since its discovery in 1934. This model presents elevated levels of total and unconjugated bilirubin in plasma and/or serum due to the absence of UGT1A1 enzymatic activity. A reduction of 87, 89 and >95% of the total plasma bilirubin level was observed 24 hours after single administration of hUGT1A1 modRNA (0.1, 0.2 and 0.5 mg/kg, respectively)-demonstrating the use of hUGT1A1 modRNA to treat maladies of elevated unconjugated bilirubin (FIG. 5C).

To confirm whether hUGT1A1 protein expressed from modRNA is correctly localized to the ER of animals treated with modRNA, liver tested using immunofluorescence. Liver samples from Gunn rats treated with 0.2 mg/kg hUGT1A1 modRNA were harvested 24 hours post i.v. injection. Correct co-localization of UGT1A1 signal (green) with the calreticulin signal (red) was shown by the merged image (yellow). The immunofluorescent images demonstrate that hUGT1A1 proteins expressed from modRNA are properly targeting the ER of hepatocytes (FIG. 5D). Calreticulin is a protein in the lumen of the endoplasmic reticulum and as calnexin it is frequently used as a marker for the ER.

### Multiple-Dose Efficacy Study in Gunn Rat Model

Multiple administration of modRNA can sustain low plasma bilirubin levels of Gunn rat animals treated with different doses of mRNA at a Q2W regimen.

Six three-week old Gunn rats/cohort were treated intravenously with hUGT1A1 modRNA at three different concentrations (0.1, 0.2 and 0.5 mg/kg) in two dosing regimens: Q2W—once every two weeks, and Q4W—once every four weeks. Animals were dosed intravenously by tail vein injection at T₀ and 14, 28, 42 and 56 days post initial treatment. Blood was obtained from submandibular or saphenous vein and collected on K3EDTA pre-coated amber tubes and centrifuged at 3,000×g for 10 minutes. Blood chemistries were analyzed for bilirubin (total and direct), alanine aminotransferase and albumin at MPI Research (Mattawan, Mich.) using an automated clinical chemistry platform (Beckman Coulter AU2700). Normal levels of total bilirubin were measured from PBS-treated wild-type animals, and as negative control Gunn rats were treated with Luciferase-modRNA Q2W at 0.5 mg/kg i.v. (highest total bilirubin levels due to lack of UGT1A1 activity).

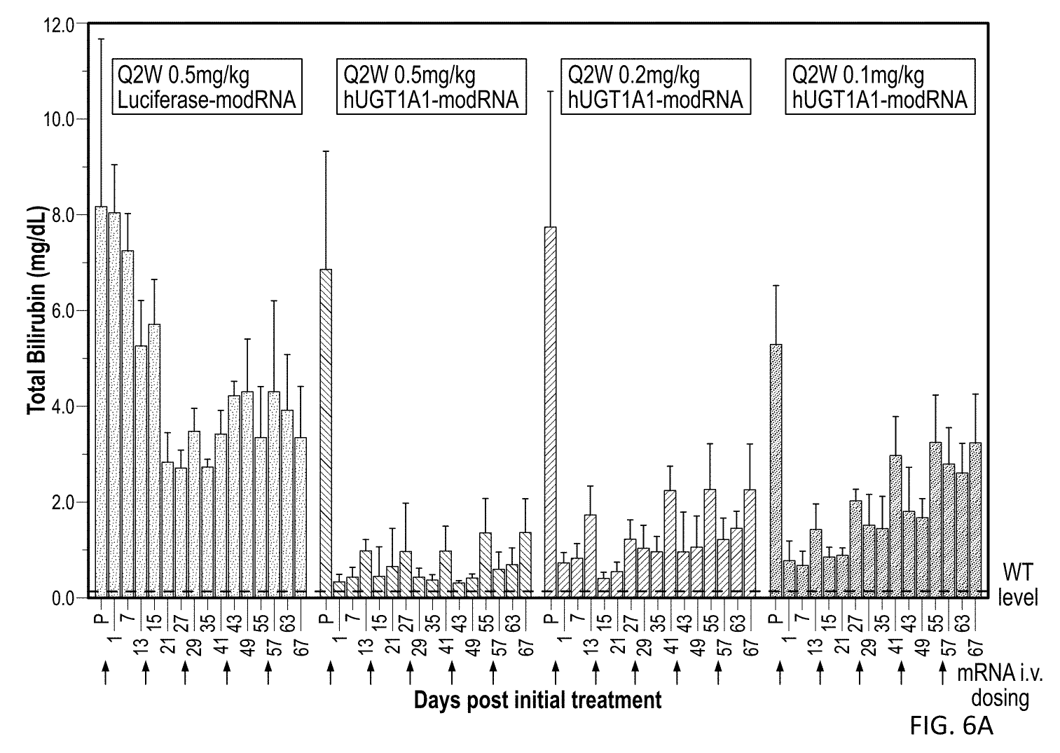

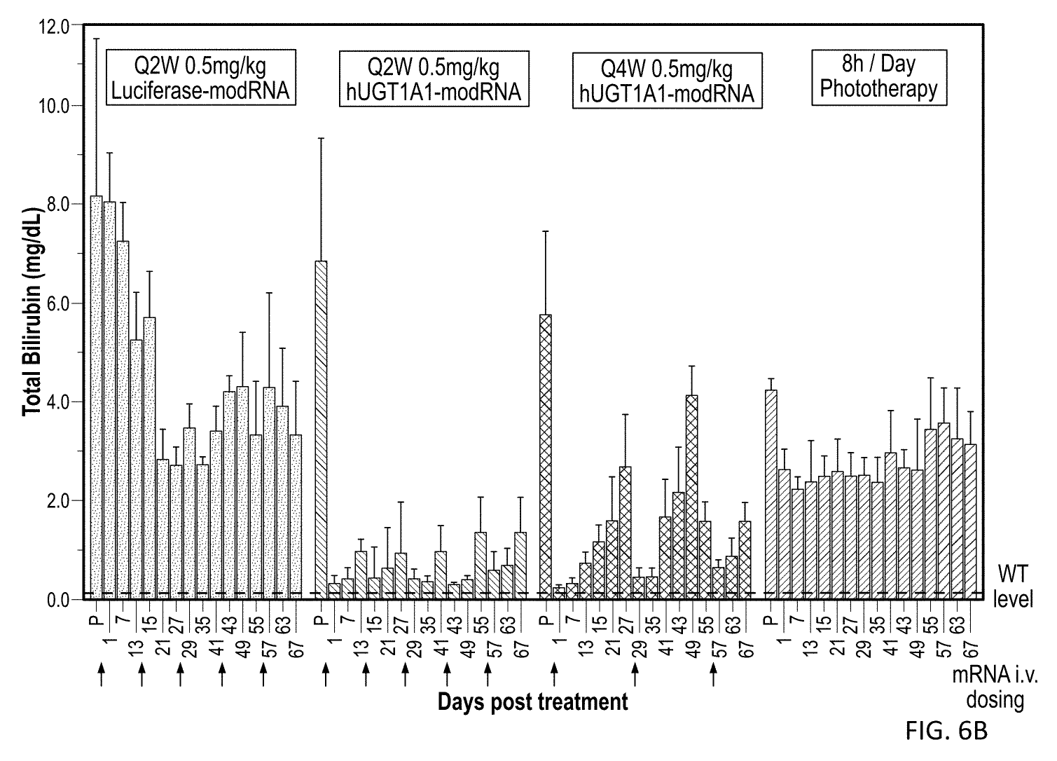

The mean values of plasma total bilirubin were remarkably reduced in UGT1A1 modRNA-treated animals at all concentrations tested. The difference between total bilirubin values in the modRNA-treated groups was statistically significant compared with the control group (Luciferase-treated animals) for at least two weeks post first treatment. All animals achieved normalization of total plasma bilirubin levels 24 h after a single i.v. administration of hUGT1A1-modRNA for 3 concentrations tested (FIG. 6A).

Long term persistent reduction of plasma total bilirubin level was observed in modRNA-treated animals in a dose-dependent fashion. The highest reduction of total plasma bilirubin was observed in the cohort treated with 0.5 mg/kg hUGT1A1-modRNA followed by 0.2 and 0.1 mg/kg measured by the area under the curve (AUC) (Table 1; FIGS. 6A and 6B).

Reduction in total plasma bilirubin levels was observed in luciferase-injected rats. The reduction observed in control group was similar to the natural reduction of total plasma bilirubin observed in naive animals confirming a natural decay on the kinetics of bilirubin after weaning age (after third week of life) (FIG. 6C).

Phototherapy is the current standard of care for CN1 patients since first week of their life. Patients with CN1 undergo 8-12 h of daily phototherapy treatment; despite such extensive exposure to blue light their total bilirubin levels do not lower to levels observed in healthy individuals. In this example the ability of phototherapy to reduce total plasma bilirubin levels was tested as a positive control in the efficacy study. There was no difference observed on the levels of total plasma bilirubin levels from animals treated with 8 h per day phototherapy and Luciferase-treated animals.

TABLE 1
Statistical Analysis of Exposures (AUC_{pre-67 days}) of different hUGT1A1-
modRNA treatment on efficacy study. AUC was calculated using total plasma
bilirubin levels from pre modRNA treatment until 2 weeks after last dose.

| | 0.5 mg/kg | 0.5 mg/kg | 0.2 mg/kg | 0.1 mg/kg | 0.5 mg/kg |
|---|---|---|---|---|---|
| | Luciferase | hUGT1A1 | hUGT1A1 | hUGT1A1 | hUGT1A1 |
| | Q2W | Q2W | Q2W | Q2W | Q4W |
| AUC_{pre-67 days (mg*day/dL)} | 46.00 | 5.25 | 14.61 | 24.62 | 20.15 |
| | 37.41 | 5.88 | 11.49 | 22.69 | 16.51 |
| | 30.12 | 10.26 | 10.20 | 25.44 | 19.81 |
| | 40.19 | 11.23 | 9.95 | 19.90 | 15.86 |
| | 38.99 | 5.06 | 9.26 | 22.05 | 11.70 |
| | — | 9.30 | — | 16.87 | 15.61 |
| Mean | 38.54 | 7.83 | 11.10 | 21.93 | 16.61 |
| SD | 5.717 | 2.748 | 2.121 | 3.158 | 3.113 |

| | 0.5 mg/kg | 0.2 mg/kg | 0.1 mg/kg | 0.5 mg/kg |
|---|---|---|---|---|
| | hUGT1A1 | hUGT1A1 | hUGT1A1 | hUGT1A1 |
| t-test | Q2W | Q2W | Q2W | Q4W |
| 0.5 mg/kg Luciferase | P < 0.0001 | P < 0.0001 | P = 0.0002 | P < 0.0001 |
| Q2W | | | | |
| 0.5 mg/kg hUGT1A1 | — | P = 0.0580 | P < 0.0001 | P = 0.0004 |
| Q2W | | | | |
| 0.2 mg/kg hUGT1A1 | — | — | P = 0.0001 | P = 0.0086 |
| Q2W | | | | |
| 0.1 mg/kg hUGT1A1 | — | — | — | P = 0.0148 |
| Q2W | | | | |

### Other Embodiments

It is understood that while the invention has been described in conjunction with the detailed description thereof, the foregoing description is intended to illustrate and not limit the scope of the invention, which is defined by the scope of the appended claims. The materials, methods, and examples are illustrative only and not intended to be limiting. All publications, patent applications, patents, sequences, database entries and other references cited and described herein are incorporated by reference in their entireties. Other aspects, advantages and modifications are within the scope of the following claims.

1. A method of treating a disease or disorder associated with a uridine diphosphate glucuronosyltransferase family 1 deficiency in a subject in need thereof, comprising administering to the subject a therapeutically effective amount of a composition comprising a modified mRNA molecule encoding a uridine diphosphate glucuronosyltransferase family 1A1 (UGT1A1) polypeptide comprising an amino acid sequence that is at least 80% identical to SEQ ID NO:4, wherein the modified mRNA molecule comprises N1-methyl pseudouridine.

2-3. (canceled)

4. The method of claim 1, wherein the amino acid sequence is at least 85% identical to SEQ ID NO:4.

5. The method of claim 1, wherein the amino acid sequence is at least 90% identical to SEQ ID NO:4.

6. The method of claim 1, wherein the amino acid sequence is at least 95% identical to SEQ ID NO:4.

7. The method of claim 1, wherein the amino acid sequence is identical to SEQ ID NO:4.

8-12. (canceled)

13. The method of claim 1, wherein the uridine diphosphate glucuronosyltransferase family 1 deficiency is type 1 Crigler-Najjar syndrome, kernicterus or hyperbilirubinemia.

14. (canceled)

15. The method of claim 1, wherein the modified mRNA molecule comprises a poly(A) tail, a Kozak sequence, a 3′ untranslated region, a 5′ untranslated region or any combination thereof.

16. A pharmaceutical composition comprising a therapeutically effective amount of a modified mRNA molecule encoding a uridine diphosphate glucuronosyltransferase family 1A1 (UGT1A1) polypeptide comprising an amino acid sequence that is at least 80% identical to SEQ ID NO:4, wherein the modified mRNA molecule comprises N1-methyl pseudouridine, and a pharmaceutically acceptable carrier, diluent or excipient.

17. A pharmaceutical composition comprising a therapeutically effective amount of a modified mRNA molecule encoding a uridine diphosphate glucuronosyltransferase family 1A1 (UGT1A1) polypeptide comprising an amino acid sequence that is at least 80% identical to SEQ ID NO:4, wherein the modified mRNA molecule comprises N1-methyl pseudouridineor active fragment thereof formulated in a lipid nanoparticle carrier.

18. A method of reducing unconjugated bilirubin levels in a subject comprising administering to the subject a therapeutically effective amount of a modified mRNA molecule encoding a uridine diphosphate glucuronosyltransferase family 1A1 (UGT1A1) polypeptide comprising an amino acid sequence that is at least 80% identical to SEQ ID NO:4, wherein the modified mRNA molecule comprises N1-methyl pseudouridine.

19-20. (canceled)

21. The method of claim 18, wherein the amino acid sequence is at least 85% identical to SEQ ID NO:4.

22. The method of claim 18, wherein the amino acid sequence is at least 90% identical to SEQ ID NO:4.

23. The method of claim 18, wherein the amino acid sequence is at least 95% identical to SEQ ID NO:4.

24. The method of claim 18, wherein the amino acid sequence is identical to SEQ ID NO:4.
